(19) United States
(12) Patent Application Publication
(10) Pub. No.: US 2022/0008751 A1
(43) Pub. Date: Jan. 13, 2022

(54) APPARATUS AND METHOD FOR GATING DELIVERY OF RADIATION BASED ON CAPACITIVE MONITORING OF RESPIRATORY MOTION

(71) Applicant: DALHOUSIE UNIVERSITY, Halifax (CA)

(72) Inventors: Parisa SADEGHI, Bedford, Nova Scotia (CA); James Robar, Halifax, Nova Scotia (CA)

(21) Appl. No.: 17/296,758

(22) PCT Filed: Dec. 6, 2019
(86) PCT No.: PCT/CA2019/051762

§ 371 Date: May 25, 2021

Related U.S. Application Data
(60) Provisional application No. 62/784,298, filed on Dec. 21, 2018.

(51) Int. Cl. A61N 5/10

(52) U.S. Cl. CPC A61N 5/1068; A61N 2005/1057

(57) ABSTRACT

An apparatus for gating delivery of radiation by a radiation delivery system to a patient is described. The apparatus includes at least one electrode positionable adjacent to but not touching a patient, at least one capacitance sensor electrically connected to the at least one electrode and configured to monitor a capacitance of the at least one electrode and generate an output signal indicative of the capacitance, and at least one processor configured to receive and process the output signal, determine a computed measure of amplitude and/or phase of respiration of the patient, and generate a gating signal for enabling or inhibiting delivery of radiation by the radiation delivery system based on the determined measure of amplitude and/or phase of respiration of the patient.

### CROSS-REFERENCE TO RELATED APPLICATION

This application claims the benefit under 35 U.S.C. § 119 of U.S. application No. 62/784,298 filed 21 Dec. 2018 and entitled CAPACITIVE MONITORING SYSTEM AND METHOD FOR REAL-TIME RESPIRATORY MOTION MONITORING which is hereby incorporated herein by reference for all purposes.

### FIELD OF THE INVENTION

The present invention generally relates to the field of radiotherapy. In particular, the present invention relates to apparatuses and methods for gating delivery of radiation to a patient for medical radiation treatment of the patient. The invention further relates to apparatuses and methods for measuring breathing, and in particular, to measuring chest movements from breathing.

### TECHNICAL BACKGROUND

External beam radiation therapy involves delivering ionizing radiation to pre-defined target locations within the body of a patient to kill target cells (e.g. cancer cells) while sparing surrounding tissues. A complicating factor is that target locations can move as a result of motions of a patient's body. For example, breathing can cause displacements of target locations. Breathing can also cause displacements of sensitive or critical non-target tissues such as, for example, the heart. External beam radiation therapy typically attempts to minimize delivery of radiation to sensitive or critical non-target tissues.

Movement of the chest caused by respiration can particularly affect radiation treatment of target tissues located in breast, lung or abdomen regions of a patient. To allow precise delivery of radiation to target locations, Deep Inspiration Breath Hold (DIBH), Deep Exhalation Breath Hold (DEBH) or gating are often used in delivering radiation treatments.

In DIBH, radiation is delivered while the patient holds their breath following a deep inhalation (e.g. a patient holds their breath while the lungs and chest have been expanded). Since the patient is instructed to hold their breath, DIBH can dramatically reduce movement of the chest during delivery of radiation. Additionally, when the patient inhales, the patient's heart is pushed in a posterior direction. DIBH can thus be used in some circumstances to position the heart further away from target locations. DIBH can be used, for example, when delivering radiation to one or more target locations within breast or lung tissue.

DEBH is similar to DIBH except that radiation is delivered while the patient holds a deep exhalation (e.g. a patient refrains from inhaling while the lungs and chest have been contracted).

Respiratory gating methods monitor a patient's breathing and inhibit delivery of radiation except in selected windows of the patient's breathing cycle (also referred to as “gating windows”). In respiratory gating methods, a radiation beam is turned on and off automatically as the patient continues to breathe during the treatment.

Known techniques for monitoring a patient's breathing include:
using a respiratory belt; and
optically monitoring movement of a patient's chest or markers attached to the patient's chest.

An example of a respiratory belt is the belt provided with the AZ-733V1 gating system available from Anzai Medical Co., Ltd. of Japan. Using a respiratory belt involves placing the respiratory belt snugly around a patient's body. As the patient breathes, the respiratory belt expands and contracts. The belt includes sensors that generate signals that vary with the patient's breathing. Respiratory belts provide output signals corresponding to movement of the patient's entire chest. Such respiratory belts cannot independently measure movement of different regions of the patient's chest. Further, respiratory belts can contact the patient. Medical equipment coming into contact with a patient may require sterilization after each use.

Optical monitors detect movement of the patient's chest by imaging or bouncing a light beam off of the patient's chest or a marker attached to the patient's chest. Such systems require an unobstructed view of the patient's chest. Optical monitoring does not work if the patient's chest is obscured by clothing, treatment devices, or other obstructions.

Various approaches to measuring a patient's breathing and/or controlling delivery of radiation during radiation treatment have been discussed in the academic and patent literature.

However, there is a general desire for alternative and/or improved methods and apparatuses for measuring a patient's breathing. There is also a general desire for alternative and/or improved methods and apparatuses for controlling delivery of a radiation treatment based on a patient's breathing and/or for gating radiation delivery to a patient, e.g. using a radiation delivery system.

### SUMMARY

In this section, a description of the general features of the present invention or disclosure is given for example by referring to possible embodiments of the invention. Specifically, various aspects of the present disclosure are described in the following. It should be noted, however, that any feature, element and/or step described in the following with respect to one aspect of the present disclosure equally applies to any other aspect of the present disclosure.

As stated above, it may be desirable to provide for an improved apparatus and method for gating radiation delivery to a patient by a radiation delivery system.

This is achieved by the subject-matter of the independent claims, wherein further embodiments are incorporated in the dependent claims and the following description.

According to a first aspect of the present disclosure, there is provided an apparatus for gating delivery of radiation, in particular ionizing radiation, to a patient by, by means of and/or using a radiation delivery system. The apparatus comprises at least one electrode positionable adjacent to but not touching a patient. The at least one electrode may be configured, formed and/or designed to be positioned in a vicinity of, close to and/or near at least a part of a body of the patient, such that the at least one electrode is spaced apart from the at least part of the body of the patient. The apparatus further comprises at least one capacitance sensor and/or capacitive sensor electrically connected to the at least one electrode and configured to determine, detect and/or monitor a capacitance of the at least one electrode and to generate an output signal indicative of the capacitance. The apparatus further comprises at least one processor configured to receive and process the output signal, determine a computed measure of amplitude and/or phase of respiration of the patient, and generate a gating signal for enabling or inhibiting delivery of radiation by the radiation delivery system based on the determined measure of amplitude and/or phase of respiration of the patient.

Generally, by monitoring, determining and/or detecting the capacitance using the at least one electrode and/or the capacitance sensor, the measure of amplitude and/or phase of respiration may be determined with high precision and/or accuracy, in particular in a fast, efficient, and contactless manner (i.e. without contacting the patient). In comparison to, for example, conventional systems employing a respiratory belt or an optical system tracking one or more markers attached to the patient's skin, a comfort can be significantly increased by the apparatus according to the present disclosure due to the contactless determination of the measure of amplitude and/or phase of respiration. Also, hygiene may be significantly improved.

The at least one processor may be configured to compute and/or calculate, based on the determined capacitance, the measure of amplitude and/or phase of respiration of the patient. Accordingly, the measure of amplitude and/or phase of respiration may be indicative, representative and/or descriptive of the amplitude and/or phase of respiration. Alternatively or additionally, the measure of amplitude and/or phase may be indicative, representative and/or descriptive of a breathing activity, a breathing state and/or a depth of inspiration of the patient.

In the context of the present disclosure, the amplitude of respiration may refer to a breathing amplitude and/or a depth of inspiration of the patient, e.g. at a certain time and/or time instant. Further, the phase of respiration may refer to a point on a breathing curve of the patient at a certain time and/or time instant, wherein the breathing curve can be given as e.g. the breathing amplitude over time.

Generally, the apparatus according to the first aspect may comprise and/or include one or more electrodes, which may face and/or may be arranged opposite to at least a part of a body of the patient that may move and/or be displaced in accordance with a respiratory movement, a respiratory motion and/or the respiration of the patient. For instance, an array of electrodes (and/or electrically conductive pads) may be arranged opposite to the at least part of the body of the patient.

Further, the capacitance of the at least one electrode may refer to a capacitance and/or capacitance value in a vicinity of the at least one electrode. Therein, the capacitance and/or the capacitance value may, for instance, refer to a capacitance between the at least one electrode and the at least part of the body of the patient arranged opposite to the at least one electrode. Alternatively or additionally, the capacitance and/or the capacitance value may refer to a capacitance between at least two electrodes of the apparatus, e.g. between at least two electrodes directly neighboring each other and/or between at least two directly neighboring electrodes.

Generally, the capacitance can be measured according to a wide range of capacitance detection methods. Example methods of capacitance detection include, but are not limited to, applying a known charge to the at least one electrode and measuring potential; applying a known potential to the at least one electrode and measuring charge; constructing an oscillating circuit whereby the frequency of that circuit depends on capacitance, and measuring frequency. In another example implementation, a capacitance bridge may be employed to measure the capacitance.

The gating signal may be indicative of whether or not radiation is to be delivered to the patient and/or a part thereof. Therein, the gating signal may, for example, refer to and/or be a binary signal, wherein a first value of the gating signal may indicate that radiation is to be delivered to the patient and a second value of the gating signal may indicate that radiation is not to be delivered to the patient. Alternatively or additionally, the gating signal may trigger emission of radiation by the radiation delivery system, e.g. by switching on a radiation source for emitting a radiation beam. It should be noted, however, that presence of the gating signal may trigger emission of radiation and absence of the gating signal may inhibit emission of radiation (or vice versa). Accordingly, the first value of the gating signal may refer to presence of the gating signal and the second value of the gating signal may refer to absence of the gating signal.

Further, the gating signal may be indicative of the radiation source of the radiation delivery system being in an on state or an off state. When the radiation source is in the on state, a radiation beam and/or radiation may be emitted by the radiation source, whereas when the radiation source is in the off state, no radiation beam and/or no radiation may be emitted. In other words, the gating signal may be indicative of whether or not the radiation beam is emitted by the radiation source.

According to an embodiment, the at least one processor is configured to compare the measure of amplitude of the respiration of the patient to a threshold and/or a threshold value for the measure and/or the amplitude) and to generate the gating signal based at least in part on whether the amplitude is equal to or greater than the threshold (and/or exceeds the threshold). In other words, the gating signal may be generated based on a comparison of the measure of amplitude of the respiration with the threshold and/or threshold value. Accordingly, if the measure of amplitude of the respiration reaches or exceeds the threshold and/or threshold value, the radiation source and/or the radiation delivery system may be switched on and/or activated, such that radiation is delivered to the patient, and if the measure of amplitude of the respiration is below the threshold and/or threshold value, the radiation source and/or the radiation delivery system may be switched off and/or deactivated, such that no radiation is delivered to the patient, or vice versa.

According to an embodiment, the at least one processor is configured to compare the measure of amplitude of the respiration of the patient to an amplitude range and to generate the gating signal based at least in part on whether the amplitude is within the amplitude range. In other words, the gating signal may be generated based on a comparison of the measure of amplitude of the respiration to the amplitude range.

According to an embodiment, the at least one processor is configured to process the output signal of the at least one capacitance sensor, to determine the measure of phase of respiration of the patient, to compare the determined measure of phase to a phase window and to generate the gating signal based at least in part on whether the measure of phase is within the phase window. In other words, the gating signal may be generated based on a comparison of the measure of phase of the respiration to the phase window. Therein, the phase window may refer to a phase range, a part of a breathing curve and/or a part of a respiratory cycle of the patient. Further, the phase window may define when in a respiration cycle and/or respiratory cycle of the patient delivery of radiation is to be enabled or inhibited. Generally, the phase window may be indicative of and/or correlate with an amplitude range and/or a range of breathing amplitudes.

According to an embodiment, the at least one electrode is substantially and/or essentially transparent to radiation deliverable by the radiation delivery system. By designing and/or configuring the at least one electrode to be substantially and/or essentially transparent to the radiation deliverable by the radiation system, absorption of the radiation by the at least one electrode can be kept at a minimum, such that the at least one electrode may substantially not interfere with and/or affect the actual radiation treatment of the patient. Also, deterioration of the at least one electrode and/or a part thereof by radiation can be kept at a minimum, thereby increasing an overall lifetime of the at least one electrode and/or the apparatus.

In the context of the present disclosure, “essentially transparent” may mean that at least 97%, preferably at least 99%, of the radiation beam can pass through the essentially transparent region and/or element of the apparatus. “Substantially transparent” may mean that at least 90% of the radiation can pass through the substantially transparent region and/or element of the apparatus.

According to an embodiment, the at least one electrode is supported and/or arranged on a substrate. Arranging the at least one electrode on a substrate may generally allow to precisely position the at least one electrode, e.g. in a desired position and/or location with respect to the at least part of the body of the patient.

According to an embodiment, the substrate is substantially and/or essentially transparent to radiation deliverable by the radiation delivery system. Also by designing and/or configuring the substrate to be substantially and/or essentially transparent to the radiation deliverable by the radiation system, absorption of the radiation can be kept at a minimum, such that the substrate may substantially not interfere with and/or affect the actual radiation treatment of the patient. Also, deterioration of the substrate and/or a part thereof by radiation can be kept at a minimum, thereby increasing an overall lifetime of the apparatus.

According to an embodiment, the substrate is made of and/or comprises a carbon fiber composite, a plastic, a plastic material or a combination thereof. Such materials may be in particular suitable substrate materials as they may be substantially and/or essentially transparent to radiation deliverable by the radiation system.

According to an embodiment, the at least one electrode is supported by a support structure that is affixable to a couch (also referred to as “treatment couch”) of the radiation delivery system. The couch may refer to a patient support and/or may be configured to support at least a part of the patient during the actual radiation treatment or radiation delivery.

According to an embodiment, the support structure comprises one or more index features configured to engage one or more index elements on the couch. At least a part of the one or more index features may be shaped in correspondence with the one or more index elements on the couch, such that the one or more index features and/or at least a part or subset thereof can engage with the one or more index elements and/or at least a part or subset thereof. For example, the one or more indexing features of the support structure may be and/or comprise one or more pins, protrusions and/or projections that are shaped, formed and/or dimensioned in correspondence with the one or more index elements on the couch. The one or more index elements may, for example, be and/or comprise one or more indexing holes, recesses, receptacles and/or notches. For example, each end of the support structure may comprise one or more index features configured to engage with one or more index elements on couch. For example, the support structure may be configured to bridge over the patient and may engage one or more index elements on either side of the treatment couch. This can allow to move the support structure and/or the at least one electrode to a desired position or location along the couch and to detachably affix the support structure to the couch in the desired position or location.

According to an embodiment, the support structure comprises a cantilever or bridge configured, formed and/or shaped to extend transversely across the couch, wherein the at least one electrode is supported by the cantilever or bridge. Such design of the support structure may allow positioning the at least one electrode at a desired position or location above the patient.

According to an embodiment, the at least one electrode comprises a plurality of electrically conductive pads (and/or conductive elements) and a switching network configured to electrically connect selected ones of the conductive pads (and/or conductive elements) in parallel to provide the at least one electrode. Accordingly, a subset of the electrically conductive pads (and/or conductive elements) may be connected in parallel to form the at least one electrode. Also, a plurality of electrodes may be formed by electrically connecting conductive pads (and/or conductive elements) of a plurality of subsets of the pads (and/or conductive elements). Therein the switching network may refer to and/or comprise a switching circuitry configured to selectively interconnect a plurality of conductive pads (and/or conductive elements). The actual selection of how many and which conductive pads (and/or conductive elements) are connected can, for example, be based on a user input.

According to an embodiment, the radiation delivery system comprises a linear accelerator. Using a linear accelerator may allow for an efficient control of radiation delivery based on the gating signal. Also, a linear accelerator may advantageously allow to adjust and/or adapt a beam energy, an energy spectrum of the radiation beam and/or a direction of the radiation beam for the radiation treatment.

According to a second aspect of the present disclosure, there is provided a method for gating delivery of radiation to a patient by a radiation delivery system. The method comprises at least the following steps:
positioning and/or arranging at least one electrode adjacent to a part of a body of the patient that moves in response to and/or in accordance with respiration of the patient, the at least one electrode being spaced apart from the part of the body to form an electrical capacitor;
determining and/or monitoring respiration of the patient by detecting, determining and/or monitoring changes in a capacitance of the capacitor; and
generating, based on the monitored respiration, a gating signal, the gating signal indicating to enable or inhibit delivery of radiation by the radiation delivery system.

According to the method of the second aspect of the present disclosure, changes in the capacitance of the at least one electrode can be detected, determined and/or monitored, and based thereon the respiration of the patient can be determined and/or monitored.

Further, the electrical capacitor may be formed between the at least one electrode and the part of the body of the patient, and the capacitance may refer to a capacitance between the at least one electrode and the part of the patient's body. Alternatively, a plurality of electrodes may be used, the capacitor may be formed between at least two directly neighboring electrodes of the plurality of electrodes, and the capacitance may refer to a capacitance between said at least two directly neighboring electrodes.

It should be noted that, in the context of the present disclosure, monitoring a measure or quantity may refer to determining the measure or quantity over time and/or over a (certain, predetermined, predefined and/or definable) period of time.

According to an embodiment, the method further comprises determining and/or monitoring changes in the capacitance by sensing and/or detecting the capacitance at a sampling frequency. The sampling frequency may, for example, be predefined, predetermined, and/or definable, e.g. by a user.

According to an embodiment, the sampling frequency is in a range between 2 Hz to 200 Hz. Such range may be suitable for accurately and precisely determining and/or monitoring the respiration of the patient, the measure of amplitude of respiration and/or the measure of phase of respiration.

According to an embodiment, monitoring and/or determining the changes in the capacitance comprises processing an output signal from a capacitive sensor and conditioning the output signal to isolate and/or determine signal components corresponding to respiration of the patient. Therein, the conditioning may be regarded as, may comprise and/or may refer to filtering the output signal. Generally, conditioning and/or filtering the output signal may allow increasing an accuracy and precision in motoring the patient's respiration.

According to an embodiment, the method further comprises generating feedback for the patient to assist the patient in controlling their breathing by providing the patient with one or more indicators based on an output signal of a capacitive sensor. Providing feedback for the patient for controlling their breathing may in particular allow enhancing effectiveness of the radiation treatment. Also, an overall dose delivered to the patient may be reduced and/or irradiation of healthy tissue that should be spared during the radiation may be avoided and/or minimized.

According to an embodiment, the feedback comprises at least one of showing the patient by how much their respiration varies from a desired respiration; reminding the patient to hold their breath at a desired point; showing the patient if they are successfully holding their breath; and letting the patient know for how much longer they are expected to hold their breath. Therein, the “desired point” may refer to a desired breathing amplitude, phase of respiration, amplitude of respiration, depth of inhalation, depth of inspiration, and/or depth of exhalation.

According to an embodiment, the method further comprises displaying the feedback using at least one of a countdown timer display, a bar indicator, a color changing indicator, one or more displays embedded in a set of glasses worn by the patient, a virtual reality head set, a portable electronic device and a display mounted within a field of view of the patient. Such means of displaying the feedback may be particularly suitable for guiding the patient during the radiation treatment, thereby ensuring effectiveness of the treatment and reducing the risk of excessive dose delivery.

According to an embodiment, the method further comprises generating the gating signal based on one or both of an amplitude and a phase of the monitored respiration.

According to an embodiment, the method further comprises determining the phase of the monitored respiration by tracking one or more trends in an output signal of the capacitive sensor. Generally, the one or more trends of the output signal may refer to one or more signal components of the output signal indicative of the patient's respiration and/or respiratory motion. Further, tracking the one or more trends may refer to determining the one or more trends over time. By tracking the one or more trends the current or actual amplitude and/or phase of respiration may be determined with high precision and accuracy. Also, tracking one or more trends may allow predicting the respiratory motion and/or respiration of the patient, e.g. at least over a certain period of time.

According to an embodiment, the method further comprises setting a gating window based on previously acquired respiration data of the patient. Therein, the previously acquired respiration data may be acquired based on monitoring the capacitance of the at least one electrode and/or based on any other means for determining respiration of the patient. The “gating window” may e.g. refer to a phase window and/or a range of amplitudes for the breathing amplitude.

According to an embodiment, generating the gating signal comprises determining whether a phase and/or an amplitude of the monitored respiration is within a gating window and dynamically adjusting and/or modifying the gating window based on the monitored respiration. Therein, adjusting the gating window may comprise increasing or decreasing a size of the gating window, wherein the size of the gating window may correspond to a part and/or period of time of a respiratory cycle of the patient. Alternatively or additionally, adjusting the gating window may comprise shifting the gating window within a respiratory cycle of the patient. By adjusting the gating window, effectiveness of the radiation treatment can be increased and the risk of excessive dose delivery can be minimized.

According to an embodiment, the at least one electrode is positioned at a minimum distance of about 1 cm to about 10 cm away from the part of the body of the patient. In other words, the at least one electrode can be positioned in a range of about 1 cm to about 10 cm away from a closest part of the body of the patient, e.g. a part of the body of the patient that is arranged closest to the at least one electrode. In such range of distances, the capacitance and/or changes in the capacitance caused by respiratory motion of the patient can be reliably detected and/or determined.

According to an embodiment, the method further comprises varying and/or increasing a signal strength of the monitored respiration by adjusting one or both of a distance of the at least one electrode from the body of the patient and an angle of the at least one electrode relative to the body of the patient. Therein, the angle of the at least one electrode relative to the body may refer to an angle enclosed by a surface normal vector of the at least one electrode and an axis of the patient's body, such as e.g. an anterior-posterior axis or a cranial-caudal axis.

According to an embodiment, the method further comprises indexing a support structure that supports the at least one electrode to a couch of the radiation delivery system. Indexing the support structure to the couch may allow to precisely position the at least one electrode e.g. at a desired position or location with respect to the patient's body.

According to an embodiment, the support structure comprises a substrate that extends transversely to the body of the patient, wherein the at least one electrode is supported by the substrate. Therein, transversely to the patient's body may mean transverse to an anterior-posterior axis and/or transverse to a cranial-caudal axis of the patient.

According to an embodiment, the method further comprises placing the at least one electrode in a location within an area corresponding to a path of a radiation beam wherein the at least one electrode and the substrate are essentially transparent to radiation of the radiation beam.

According to an embodiment, monitoring changes in the capacitance of the capacitor comprises applying a direct current (DC) signal to the at least one electrode.

According to an embodiment, the at least one electrode comprises a plurality of electrodes, each of the plurality of electrodes located proximate to a different part of the body. Using a plurality of electrodes, each located proximate to a different part of the body, may allow determining and/or monitoring the respiration with high precision. Also, this may allow differentiating between movements of the patient's body caused by respiration and other movements of the patient.

According to an embodiment, the method further comprises electrically connecting a plurality of the electrodes, conductive pads and/or conductive elements in parallel. Connecting a plurality of electrodes may allow detecting movements of a larger part of the patient when compared to using only a single electrode. In turn, this may lead to an increased signal strength and/or an increased precision in determining the patient's respiration, amplitude of respiration and/or phase of respiration.

According to an embodiment, the method further comprises placing a conductive material on a surface of the body opposite the at least one electrode. This may lead to an increased signal strength of the at least one electrode and to an increased precision in determining the respiration, the amplitude of respiration and/or the phase of respiration.

According to a third aspect of the present disclosure, there is provided an apparatus for gating delivery of radiation to a target tissue within a body of a patient. The apparatus comprises an imobilizable substrate; a capacitor formed by the body of the patient and an electrode supported by the substrate, the electrode being positionable adjacent a region and/or part of the body movable by respiration of the patient, the electrode being spaced apart from the body; a capacitive sensor configured to receive a signal from the electrode corresponding to an amount of electric charge on a surface of the electrode and to generate an output signal representative of a distance by which the body of the patient is separated from the electrode; and a processor configured to:
receive an output signal of the capacitive sensor;
process the output signal to measure and/or determine respiration of the patient; and
generate a gating signal to enable or inhibit delivery of radiation to the target tissue by comparing measured respiration to a gating window, the gating window defining when in a respiration cycle and/or respiratory cycle of the patient delivery of radiation is to be enabled or inhibited.

The present disclosure also relates to the use of any of the first to third aspect for medical treatment of a patient. Particularly, the present disclosure also relates to the use of the apparatus according to the first aspect and/or the third aspect for medical treatment of a patient.

A fourth aspect of the present disclosure relates to the use of a capacitive sensor for monitoring respiration, an amplitude of respiration and/or a phase of respiration for gating delivery of radiation to a patient by a radiation delivery system.

It is emphasized that features, functions, elements and/or steps, which are described above and in the following with reference to one aspect of the invention or disclosure, equally apply to any other aspect of the invention or disclosure described above and in the following. Particularly, features and/or elements, as described above and in the following with reference to the apparatus according to the first aspect, equally apply to the method according to the second aspect, and/or the apparatus according to the third aspect, and vice versa.

Briefly summarizing, the present disclosure includes, without limitation:
methods and apparatus for measuring a patient's breathing;
methods and apparatus for measuring movement of a patient's chest;
methods and apparatus for controlling delivery of a radiation treatment based on a patient's breathing;
radiation treatment systems;
contactless capacitive electrode systems.

It is emphasized that the invention or disclosure as described with reference to any of the first to fourth aspect does not need to involve or in particular comprise or encompass an invasive step which would represent a substantial physical interference with the body requiring professional medical expertise to be carried out and entailing a substantial health risk even when carried out with the required professional care and expertise. For example, these aspects of the invention do not comprise a step of positioning a medical implant in order to fasten it to an anatomical structure or a step of fastening the medical implant to the anatomical structure or a step of preparing the anatomical structure for having the medical implant fastened to it. More particularly, these aspects of the invention do not involve or in particular comprise or encompass any surgical or therapeutic activity. These aspects of the invention are instead directed as applicable to any non-invasive medical application. For this reason alone, no surgical or therapeutic activity and in particular no surgical or therapeutic step is necessitated or implied by carrying out the invention.

The methods and apparatus described herein may optionally be used in conjunction with surgical and/or therapeutic activities.

Further aspects and example embodiments are illustrated in the accompanying drawings and/or described in the following description.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIGS. 1A and 1B are schematic illustrations of example cross-sections of a patient's chest.

FIG. 2 is a schematic illustration of apparatus according to an example embodiment which includes a capacitor formed by positioning an electrode adjacent to a patient's chest.

FIG. 3 is a schematic illustration of example movement of a patient's chest as the patient breathes.

FIG. 4 is a schematic illustration of an example capacitive sensing circuit.

FIG. 5 is a partial plan view of an example chest region of a patient.

FIG. 6A is an example continuous time signal of measured capacitance.

FIG. 6B is an example discrete time signal of measured capacitance.

FIG. 6C is an example control signal.

FIG. 7 is a flow chart illustrating a method according to an example embodiment.

FIG. 8 schematically illustrates a radiation treatment system according to an example embodiment.

FIG. 8A is a plan view of a treatment couch according to an example embodiment.

FIGS. 9A to 9G illustrate electrodes according to example embodiments.

FIGS. 10A and 10B illustrate electrodes according to example embodiments.

FIGS. 11A to 11J illustrate supporting substrates for supporting electrodes according to example embodiments.

FIG. 12 schematically illustrates a graphical user interface according to an example embodiment.

FIGS. 13A and 13B schematically illustrate a feedback interface according to an example embodiment.

### DETAILED DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive sense.

Aspects of the present technology monitor a state of respiration of a patient by applying measurements of capacitance between an electrode placed near the patient and the body of the patient. Motions of the patient's body (e.g. motions resulting from respiration) vary the distance between the surface of the patient's body and the electrode. This, in turn, alters the capacitance. By monitoring the capacitance one can:
determine whether or not the patient's body is moving (e.g. determine whether the patient has stopped holding their breath);
determine when the patient's body is in a reference state (e.g. a gating window); and/or
monitor the patient's respiration over time.
   In some embodiments the electrode and any support for the electrode are essentially transparent to treatment radiation.

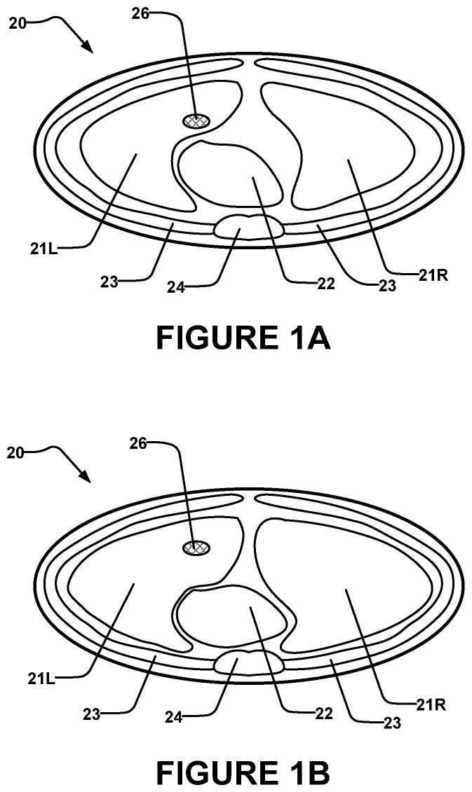

FIGS. 1A and 1B provide a non-limiting illustration of a case where the present disclosure and/or the technology described therein may be applied. FIG. 1A is an example schematic cross section of a chest 20 of a patient P. Chest 20 comprises ribs 23 and spinal vertebrae 24. Chest 20 also comprises a left lung 21L and a right lung 21R. Left lung 21L comprises target tissue 26 (e.g. a tumour to be treated). Target tissue 26 may, for example, be treated by radiation treatment.

As shown in FIG. 1A, target tissue 26 is proximate to heart 22 when lung 21L is deflated (i.e. when air has been partially, or fully, exhaled from lung 21L). As air is inhaled into lung 21L, the distance between target tissue 26 and heart 22 increases (see FIG. 1B). Exposure of sensitive and/or critical non-target tissues (e.g. heart 22) to radiation may be reduced, or minimized, by delivering radiation to target tissue 26 during a state of the patient's breathing when target tissue 26 is farthest away from the sensitive and/or critical non-target tissues. It is therefore desirable to gate delivery of the radiation so that radiation is delivered in the phase of respiration corresponding to FIG. 1B and to inhibit delivery of radiation in other phases of respiration (including the phase corresponding to FIG. 1A). In cases where the phase of respiration corresponding to FIG. 1B corresponds to an inhaled or exhaled respiration phase, radiation may be delivered during DIBH or DEBH.

The present disclosure and/or the technology described therein may be applied to gate delivery of radiation based on a patient's state of breathing. An electrically conductive electrode is placed adjacent to a patient's body (e.g. adjacent to the patient's chest and/or abdomen) without touching the patient's body. The patient's body is naturally electrically conductive. Placing the electrode adjacent to the patient's body forms a capacitor. Alternatively or additionally, a capacitor may be formed between at least neighboring and/or directly neighboring electrodes. The capacitance of the capacitor varies with the distance between the electrode and the patient's body. The electrode may be placed adjacent to a relevant part of the patient's body (e.g. adjacent to the patient's chest or abdomen).

As the patient breathes, the patient's lungs expand and contract and the outer surface of the patient's body moves relative to the electrode. These movements alter the capacitance of the capacitor. Changes in capacitance may be monitored to determine a state of the patient's breathing. Delivery of radiation to patient P may be gated based on the determined respiratory state.

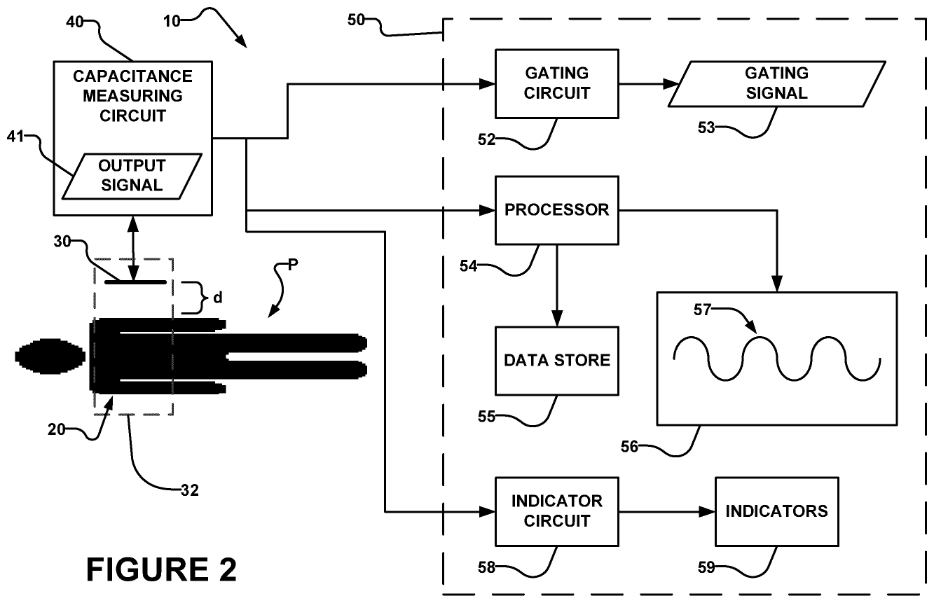

FIG. 2 illustrates a capacitor 32 formed by placing an electrode 30 adjacent to chest 20 of patient P. A distance d separates electrode 30 from chest 20. Capacitance C of capacitor 32 is inversely proportional to distance d. Capacitance C may, for example, be approximated as follows:

$$C = \frac{ɛ A}{d} \qquad ( 1 )$$

where A represents the surface area of capacitor 32 and s represents a dielectric constant of the material between electrode 30 and chest 20. Typically, a surface area of electrode 30 is substantially smaller than a surface area of the patient's body and so the area A of capacitor 32 may be taken to be equal to the surface area of electrode 30.

Capacitance C of capacitor 32 varies with changes in the distance d separating electrode 30 from chest 20 (and/or separating at least two electrodes forming the capacitor). If electrode 30 is fixed, distance d changes as patient P breathes. Thus, capacitance C also changes as patient P breathes. Capacitance C may be monitored by a capacitance measuring circuit 40 as described in more detail below.

An output signal 41 of capacitance measuring circuit 40 is supplied to processing circuits 50 that may apply output signal 41 in various ways. FIG. 2 illustrates three example applications. For one application output signal 41 is processed by a gating circuit 52 to yield a gating signal 53. Gating signal 53 may be supplied to inhibit or permit delivery of radiation from a radiation source. For example, gating circuit 52 may set the gating signal to permit delivery of radiation from the radiation source if output signal 41 satisfies a predetermined criterion (for example, output signal 41 being within a selected range) and to inhibit delivery of radiation from the radiation source otherwise. Gating signal 53 may be a logic and/or binary signal that has one value corresponding to ‘inhibit radiation delivery’ and another value corresponding to ‘permit radiation delivery’.

This application may be used to gate delivery of radiation to coincide with a desired phase of the patient's respiration cycle (e.g. fully inhaled or fully exhaled), or within a predetermined range of a reference phase etc.

For another application, a processor 54 is connected to one or both: store in a data store 55 and display as a trace 57 on a display 56 a time series of values of output signal 41 (or a result obtained by processing output signal 41).

This application may be used to observe the patient's respiratory cycle, determine appropriate criteria for gating, as a visual aide to training patients to breathe in a certain fashion (e.g. DIBH or DEBH) and/or to compare a patient's current and previous respiration patterns.

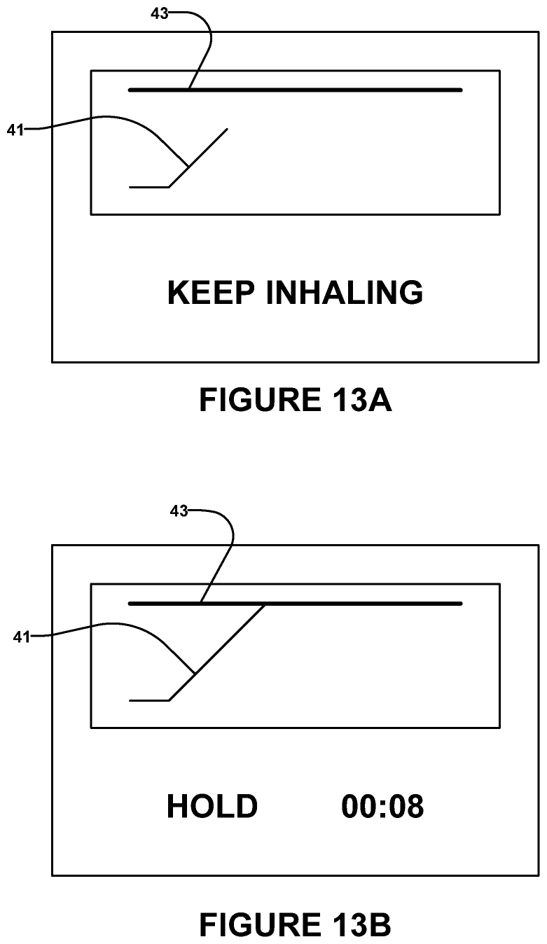

For another application an indicator circuit 58 controls one or more indicators 59 in response to output signal 41. In an example case, indicator circuit 58 controls indicators 59 to indicate one or more of:
by how much and in what direction does the value of output signal 41 differ from a desired value/criterion 43 (see e.g. FIG. 13A);
for how long has the value of output signal 41 been in a desired range.
   Indicator circuit 58 may, for example, comprise a timer that is triggered when output signal 41 satisfies a desired value/criterion 43 (see e.g. FIG. 13B). Indicators 59 may, for example, comprise one or more of a countdown timer display, a bar indicator, a color changing indicator or the like. In some embodiments, one or more indicators 59 are displayed to patient P. Indicators 59 may, for example, be provided on a display (or displays) embedded in a set of glasses worn by patient P, a virtual reality headset, a portable electronic device, a display mounted to be within a field of view of patient P, or the like.

This application may be used to remind a patient to hold their breath at a desired point, to show the patient if they are successfully holding their breath at the desired phase and/or to let the patient know for how much longer they are expected to hold their breath.

Where processing circuits 50 are constructed to perform two or more of the above applications, the circuitry for performing the different applications may optionally be combined. For example, a processor 54 may be configured by way of suitable software to perform two or more of the above applications.

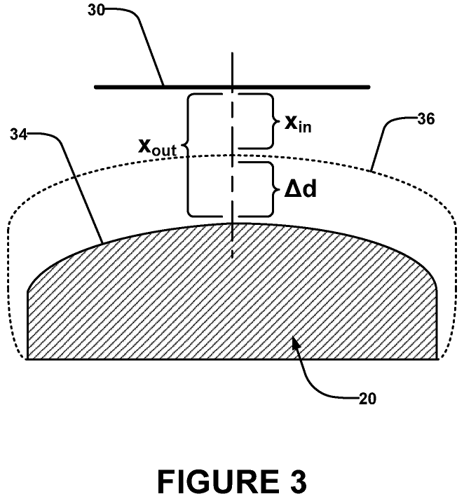

FIG. 3 schematically illustrates example movement of chest 20 relative to stationary electrode 30 as patient P breathes. Solid line 34 illustrates an outer surface of chest 20 corresponding to a contracted state of chest 20 (i.e. air has been exhaled from patient P's lungs). Solid line 34 is separated from electrode 30 by a distance xₒᵤₜ. Dashed line 36 illustrates an outer surface of chest 20 corresponding to an expanded state of chest 20 (i.e. air has been inhaled into patient 10's lungs). Dashed line 36 is separated from electrode 30 by a distance xᵢₙ. A change in distance d may be represented by:

Δd=xₒᵤₜ−xᵢₙ (2).

If a time-varying position of chest 20 relative to electrode 30 is represented as x(t), a corresponding time-varying capacitance C(t) may, for example, be represented as:

$$C ( t ) = \frac{F}{x ( t )} \qquad ( 3 )$$

where F is a proportionality factor that is constant, at least to a first approximation.

A surface area of electrode 30 may be chosen to be large enough for capacitance C to be measurable. The surface area of electrode 30 may also be selected to be small enough that changes in capacitance result primarily from movements of a desired part of the patient's body (e.g. a part of the chest or a part of the abdomen). Additionally, the surface area of electrode 30 may be large enough to average out effects of local differences in distance d that arise from the fact that the body of patient P is not perfectly smooth or parallel to electrode 30.

In some embodiments electrode 30 has a surface area in the range of about 4 cm² to about 900 cm².

In some embodiments, capacitance C may be increased by inserting a dielectric material between electrode 30 and the body of patient P and/or between at least two electrodes forming the capacitor (i.e. a material having a higher dielectric constant value E than air). In some embodiments, the dielectric material conforms to patient P's body. The dielectric material may, for example, take the form of a blanket. In some embodiments, the dielectric material is made of a plastic, foam or the like. In some embodiments, the dielectric material is easily sterilized. The dielectric material is advantageously essentially transparent to radiation of the type to be delivered to patient P.

Apparatus 10 includes means for holding electrode 30 close to a region of interest of patient P's body. For example, if movement of chest 20 is of interest, electrode 30 may be held adjacent to chest 20 (e.g. for treatment of lung tumours). FIG. 5 shows an example region 28 in chest 20. As another example, if movement of an abdominal region of patient P is of interest, electrode 30 may be held adjacent the abdomen (e.g. for treatment of liver tumours). Ideally the holding means is flexible enough to allow electrode 30 to be positioned at selected locations near to patients having varying body shapes and sizes. In some cases it may be desirable to locate electrode 30 away from devices that may affect capacitance measurements such as, for example, a pace maker.

As another example, different patients may breathe differently. Some patients may breathe by diaphragmatic breathing (also called abdominal breathing or belly breathing). For such patients it may be desirable to have at least one electrode 30 positioned near the patient's abdomen to best monitor respiration. Other patients may breathe primarily by chest breathing. For such patients positioning an electrode 30 near the patient's chest can facilitate monitoring the patient's respiration.

Advantageously, electrode 30 does not contact the body of patient P. Electrode 30 may be held adjacent the body of patient P at any distance d that produces a capacitance C that is measurable by capacitance measuring circuit 40. In some embodiments, electrode 30 is held a minimal distance d away from chest 20 to maximize capacitance C. In some embodiments, electrode 30 is positioned in the range of 1 cm to 10 cm (e.g. about 5 cm) away from the closest part of the body of patient P.

Where apparatus as described herein is used in conjunction with radiation treatment it is desirable that electrode 30 and/or any structure supporting electrode 30 should not interfere with the radiation treatment by blocking or significantly altering a radiation beam. This goal may be achieved by either or both of positioning electrode 30 so that neither electrode 30 nor any structure supporting electrode 30 is in the way of the radiation beam and/or by making electrode 30 and relevant parts of a supporting structure so that they are essentially transparent to the radiation of the radiation beam to be applied to the patient, at least in a region through which the radiation beam will pass through electrode 30 and/or its supporting structure. “Essentially transparent” means that at least 97%, preferably at least 99% of the radiation beam passes through the essentially transparent region. In some embodiments electrodes and/or structures are substantially transparent to radiation. “Substantially transparent” means that at least 90% of the radiation passes through.

In some embodiments, the radiation beam comprises a photon beam. In such embodiments, attenuation of the photon beam as it passes through an attenuator (e.g. electrode 30 and/or any structure supporting electrode 30) may, for example, be approximated as follows:

N=N₀e^{-μx} (4)

where N₀ represents a number of photons incident on the attenuator, N represents a number of photons exiting the attenuator, x is a thickness of the attenuator and μ is a linear attenuation coefficient of the attenuator. In example cases where it is desired for at least 99% of the radiation beam to pass through the attenuator, μx should equal at most 0.01005.

In some embodiments electrode 30 is made essentially transparent to radiation to be delivered to a patient (e.g. a photon beam, a proton beam, etc.) by making electrode 30 very thin. For example, electrode 30 may comprise a thin film of a metal or other electrical conductor. In some embodiments electrode 30 is made of a thin film of copper or aluminum. In some embodiments, a desired value (or range of values) for μx described above in association with Equation 4 is used to set a maximum thickness (or range of thicknesses) of electrode 30 in order to achieve a desired radiation transparency.

Additionally, or alternatively, reducing one or more of an atomic number, density, electron density, or the like of a material that is used to make electrode 30 may increase the transparency of electrode 30 to radiation.

All or parts of a structure supporting electrode 30 may be made to be essentially transparent to a radiation beam. This may be achieved through suitable choice of materials and/or by making the materials thin in a direction through which the radiation passes. For example the structure may be made from one or more materials such as a carbon fibre composite or a plastic (e.g. Lucite) or a carbon fibre shell with a low density core that has a low coefficient of absorption for the radiation of the radiation beam and which tends not to scatter the radiation beam. In some embodiments, the structure is rigid. In some embodiments, the structure is made of a low density material. In some embodiments, the structure comprises carbon fiber sheets, one or more shells enclosed by a rigid layer having a foam core, hexagonal-filled low density sheets, a 3D printed lattice, or the like. The structure may be made to be thin in a direction of propagation of the radiation beam.

In some embodiments, electrode 30 is electrically isolated and/or shielded to prevent electric charge from leaking out of electrode 30. The structure is preferably electrically insulated from electrode 30. For example, a layer of an electrical insulator may be provided between electrode 30 and the structure supporting electrode 30.

Optionally, electrode 30 is supported on or encapsulated within a supporting substrate 70. For example, electrode 30 may be supported on a sheet of plastic or a carbon fibre composite. In some embodiments, electrode 30 is sandwiched between two layers of a laminated structure. In embodiments where electrode 30 is supported on one face of a sheet or other support panel, the exposed surface of electrode 30 (opposed to the sheet) may be coated with a protective layer. For example, exposed portions of the surface of electrode 30 may be covered with an electrically insulating material. In some embodiments, electrode 30 is supported on a face of the sheet or other support panel which is closest to the patient.

There are a wide range of possible structures for supporting an electrode 30. Such structures may be fixed or may provide adjustment for the position and/or angle of electrode 30. In most radiation treatment contexts the patient receives radiation while the patient is supported on a radiation treatment couch. In many cases the patient is treated while in a supine position. There are some cases where the patient may be treated while in a prone or other position on the radiation treatment couch.

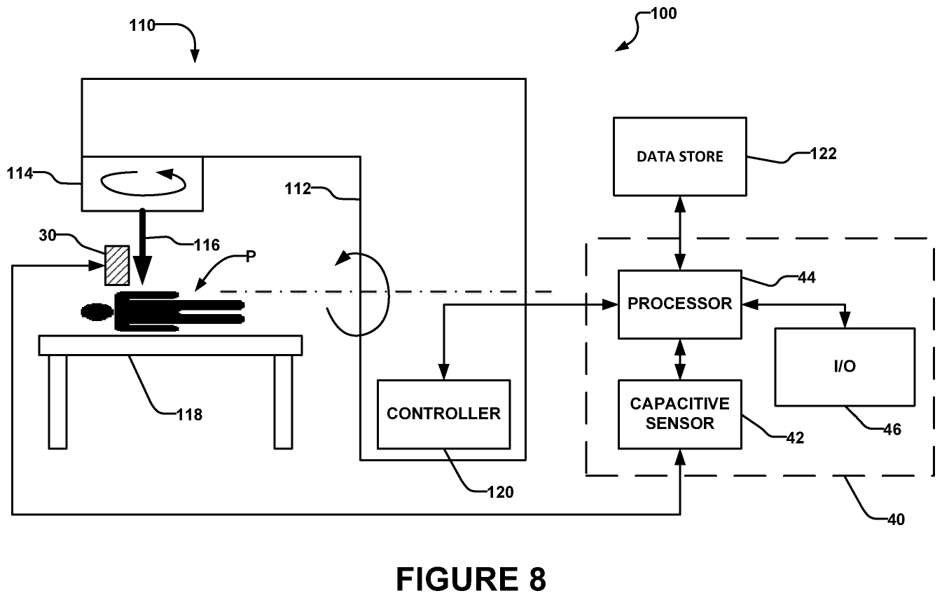

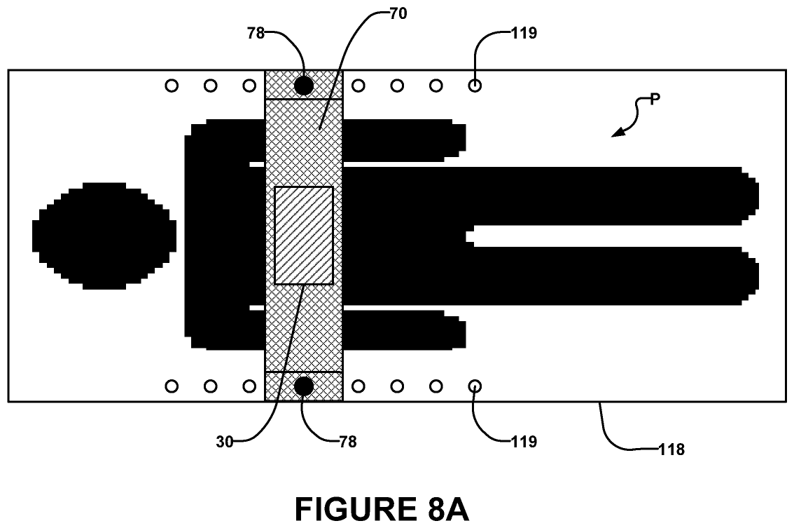

In some embodiments, a support structure is fixed relative to a treatment environment. For example, the support structure may be indexed to treatment couch 118 (see e.g. FIG. 8A), a wall of a treatment room, a floor of a treatment room or the like. In some embodiments, the support structure may be selectively immobilized at different locations relative to the treatment environment. For example, each longitudinal extending edge of a treatment couch 118 may include a plurality of bores 119 or other indexing features for receiving one or more fasteners 78 (see e.g. FIG. 8A). A location of support structure may be selected based on a region of interest for monitoring the patient's respiration as well as a posture of the patient on the radiation treatment couch.

In some embodiments the support structure for electrode 30 is configured to securely and repeatably be engaged to the radiation treatment couch. For example, the support structure may include indexing features such as pins or other projections shaped and dimensioned to engage indexing holes or notches provided on a radiation treatment couch or a rail attached to a radiation treatment couch. In some embodiments, each end of the support structure may simultaneously engage a plurality of indexing holes, notches or the like of the treatment couch. For example, the support structure may be configured to bridge over the patient and may engage indexing elements on either side of the treatment couch.

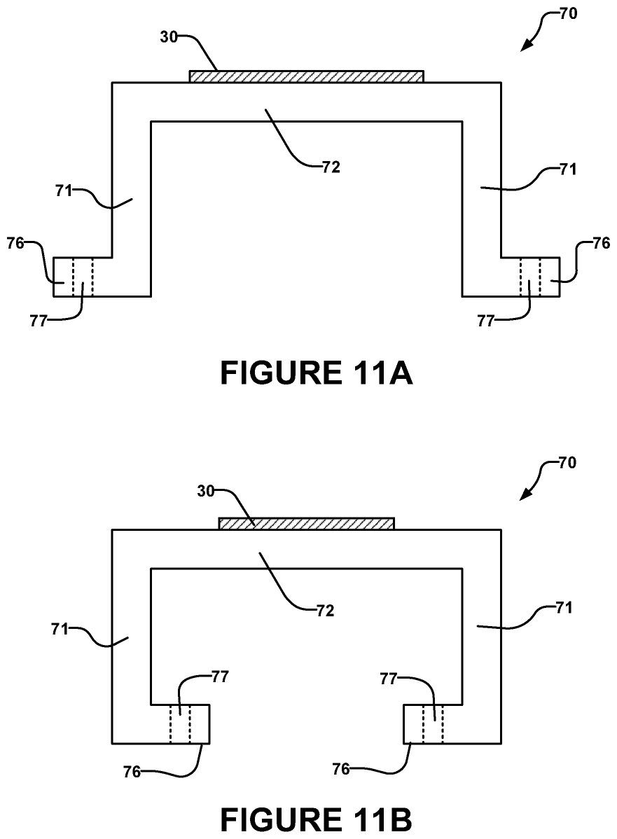

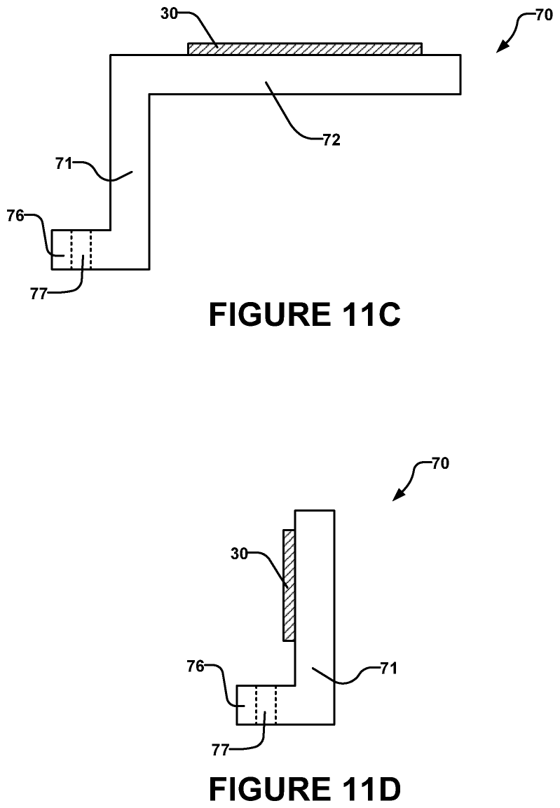

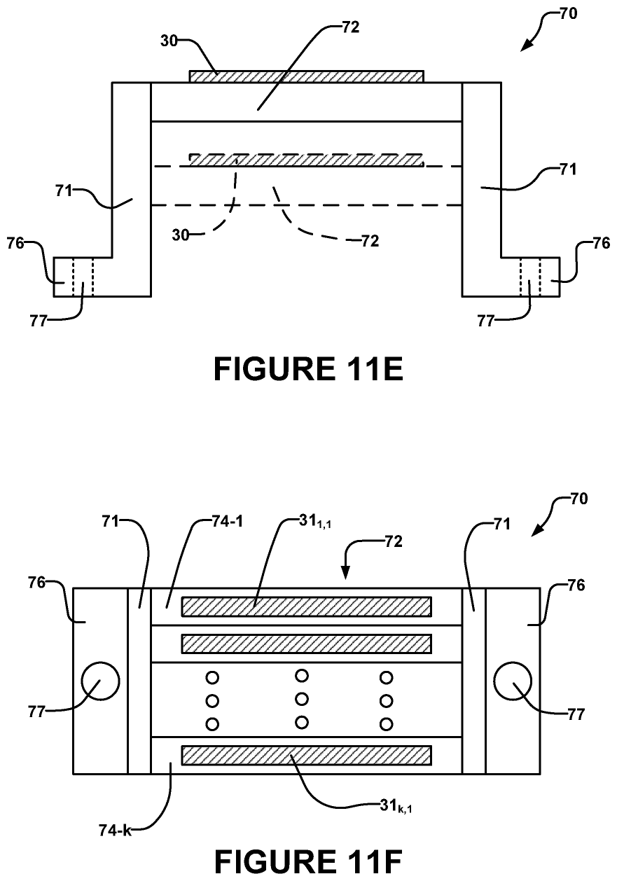

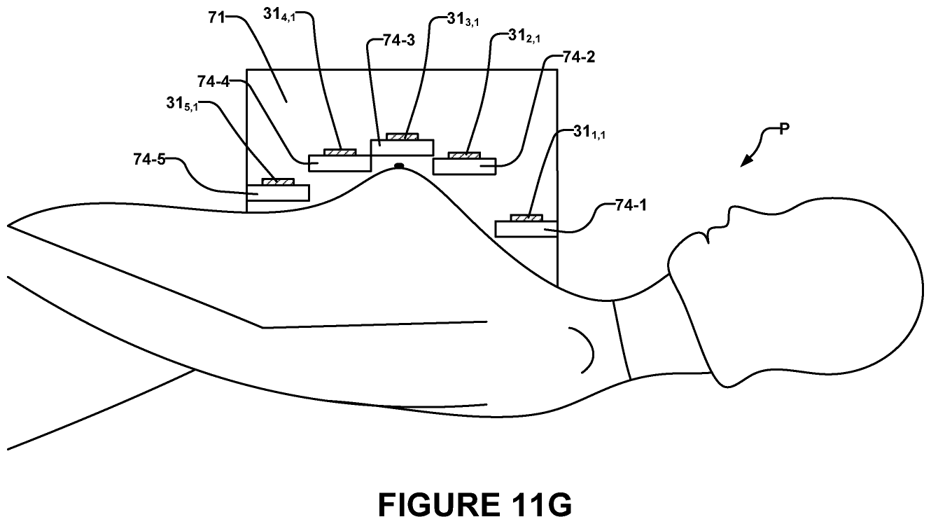

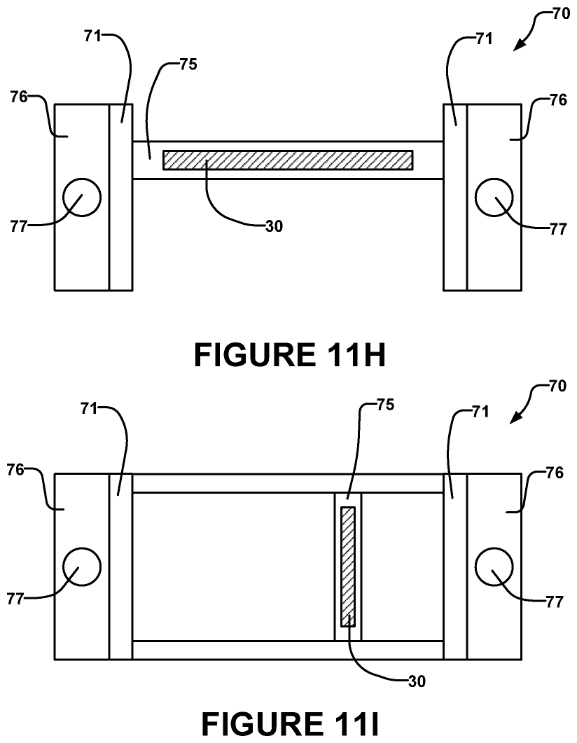

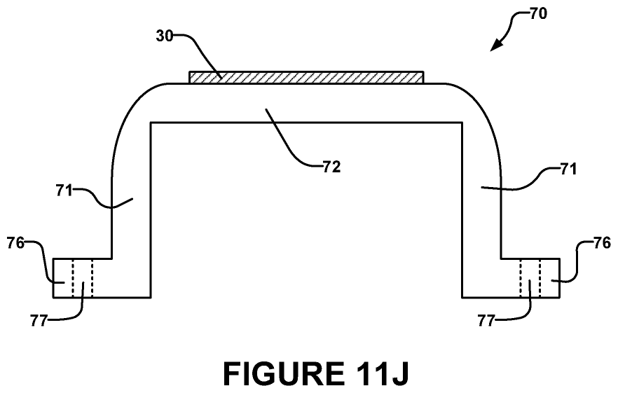

FIGS. 11A and 11B show example embodiments in which a support structure 70 includes feet 76 defining bores 77. Fasteners 78 pass though bores 77 and corresponding bores 119 of treatment couch 118 (see e.g. FIG. 8A). In some embodiments, a foot 76 comprises a plurality of bores 77.

In some embodiments electrode 30 is supported by a member such as an arch or cantilever arm that extends over the body of patient P. The arch or arm may be configured to attach to a treatment couch or to an immobilizing accessory to a treatment couch (e.g. a breast board or the like). Since, as mentioned above, electrode 30 can be used to detect respiration at a range of distances from a patient's body the member is not necessarily adjustable. Different sizes of electrode support (e.g. small, medium, large) may be provided for patients of different sizes. Each electrode support may accommodate a range of patient sizes.

FIG. 11A shows a side view of an example support structure 70. Support structure 70 comprises side supports 71 and beam 72. In the example embodiment shown in FIG. 11A, beam 72 supports electrode 30. In some embodiments, support structure 70 comprises a single side support 71 (i.e. one end of beam 72 is unsupported; see e.g. FIG. 11C).

It is generally desirable for the support structure to be reasonably close to the patient's body so that it does not physically interfere with positioning a radiation source at a desired location or with moving the radiation source on a desired trajectory. A spatial footprint of support structure 70 may be minimized. For example, support structure 70 may comprise rounded corners (see e.g. FIG. 11J).

In some embodiments, an electrode 30 is supported by a side support 71 (see e.g. FIG. 11D). Such an electrode 30 may sense lateral expansion and contraction of a patient's chest as the patient breathes in and out.

In some embodiments, as shown by dashed lines in FIG. 11E, a position of beam 72 relative to side supports 71 may be adjustable (i.e. a height of beam 72 relative to a bottom of support structure 70 may be adjusted to suit a particular patient). In such embodiments, proximity of electrode 30 relative to chest 20 is adjustable. In some embodiments, the position of beam 72 may be varied to account for different shapes and/or sizes of patient P. Additionally, or alternatively, a height of beam 72 may be reduced to increase the measured capacitance for more accurate determination of respiration.

Some regions of chest 20 may expand and/or contract more than other regions of chest 20 as a patient breathes. For example, an anterior surface of chest 20 may expand and/or contract more than a side surface of chest 20. In such cases, changes in capacitance C based on breathing may be different based on where electrode 30 is positioned relative to chest 20. In some embodiments, electrode 30 is positioned to maximize differences in capacitance C corresponding to expansion and contraction of chest 20. For example, electrode 30 may be positioned adjacent a region of chest 20 where Δd is observed to be larger than in other regions.

In some embodiments, beam 72 comprises a movable member 75 as shown in FIGS. 11H and 11I. Movable member 75 supports electrode 30. Movable member 75 may be used to position electrode 30 in a cranial-caudal direction (see e.g. FIG. 11H) or a sinister-dexter direction (see e.g. FIG. 11I) relative to a patient. Indicia may be provided to allow electrode 30 to be repeatedly moved to a desired position. Such indicia may be useful where a patient receives radiation in multiple sessions that are spaced apart and it is desired to monitor the patient's respiration using the same electrode location in each session.

In circumstances in which patient P is lying in a prone position on treatment couch 118 (e.g. for treatment of rectal cancer), treatment couch 118 may comprise a belly board. The belly board may comprise a bore for receiving at least a portion of chest 20 of patient P. Electrode 30 and/or supporting substrate may be positioned on a bottom surface of the belly board (or treatment couch 118) to measure respiration related movements of patient P.

Apparatus as described herein is not limited to having a single electrode 30. The apparatus may include two, three or more electrodes. Embodiments which include plural electrodes may, for example, exploit the plural electrodes in one or more of the following ways:
capacitance between the patient's body may be monitored independently for each of two or more electrodes to provide redundant information regarding the patient's respiration. The redundant monitoring may be performed using the same capacitance monitoring circuit or by plural redundant capacitance monitoring circuits. The redundant information may be applied to provide a more accurate gating signal (for example, the gating signal may be set to enable delivery of radiation only if the monitored capacitance from two or more electrodes or, in some embodiments, all of three or more electrodes, agree that the patient's respiration is in a desired window for delivery of radiation);
two or more electrodes may be connected in parallel to create a larger electrode. In some embodiments the shape and/or size of the larger electrode may be tailored to match the shape and/or size of a part of the body that it is desired to monitor;
one or more of a plurality of electrodes may be selected for being in a desired location relative to the patient. For example, one or more electrodes that are located proximate a part of the patient's body that is of particular interest or part of the patient's body that moves a desired amount when the patient breathes may be selected;
two or more electrodes may be selected to simultaneously measure lateral and anterior-posterior movement of the patient's body as the patient breathes. For example, one electrode (or a plurality of electrodes) may be positioned adjacent a lateral region of the patient's body to measure lateral movement of the patient's body (e.g. such electrode(s) may be supported by a side support 71). A second electrode (or second plurality of electrodes) may be positioned adjacent an anterior or posterior region of the patient's body to measure anterior-posterior movement of the patient's body (e.g. such electrode(s) may be supported by beam 72); and
capacitance between at least two electrodes may be determined and/or monitored in order to determine and/or monitor respiration of the patient, the measure of amplitude of respiration, and/or the measure of phase of respiration.

In embodiments which provide plural electrodes, a switching network and/or switching circuitry may be supplied to allow connection of selected one(s) of the electrodes to one or more capacitance monitoring circuits individually and/or in parallel combinations. In some embodiments, the switching network comprises a multiplexer.

Plural electrodes may be spaced apart from one another. For example, in some embodiments, plural electrodes are spaced apart by distances in the range of ½ cm to 5 cm (preferably about 2½ cm).

In embodiments which provide plural electrodes the relative positions of the plural electrodes may be fixed (for example, the plural electrodes may be supported at different locations on the same substrate). In some embodiments the support structure may be constructed to allow the relative positions of some plural electrodes to be adjusted.

For example, FIG. 11F shows an example embodiment in which a beam 72 comprises a plurality of segments 74-1, . . . 74-k (collectively segments 74). Each of the plurality of segments 74 supports an electrode 31. Each of the plurality of segments 74 is adjustable in height independent of the remaining segments 74 in the plurality. In such embodiments, each segment 74 may be positioned at a different height to maintain a consistent distance d between each of the segments 74 and chest 20. For example, a segment 74-3 positioned adjacent breast tissue may be positioned higher than a segment 74-5 positioned adjacent the patient's abdomen (see e.g. FIG. 11G). In some embodiments electrodes 31 carried by two or more segments 74 are electrically connected to effectively form a single electrode 30 which can be connected to a capacitance monitoring circuit (e.g. circuit 40 described elsewhere herein).

In some embodiments, an angular orientation of each of the plurality of segments 74 relative to patient P is independently adjustable. Varying the angular orientation may, for example, facilitate detection of motion of patient P's body in one or more selected directions as patient P breathes.

During a course of radiation treatment being delivered by radiation treatment system 100 (see e.g. FIG. 8), radiation beam 116 may need to pass through one or both of electrode 30 and supporting substrate 70. Attenuation of radiation beam 116 would change a radiation dose that is delivered to patient P. In some embodiments, one or both of electrode 30 and supporting substrate 70 are at least partially transparent to radiation beam 116. In some embodiments, one or both of electrode 30 and supporting substrate 70 minimally attenuate radiation beam 116. In some embodiments, the combination of electrode 30 and supporting substrate 70 is essentially transparent to radiation of beam 116.

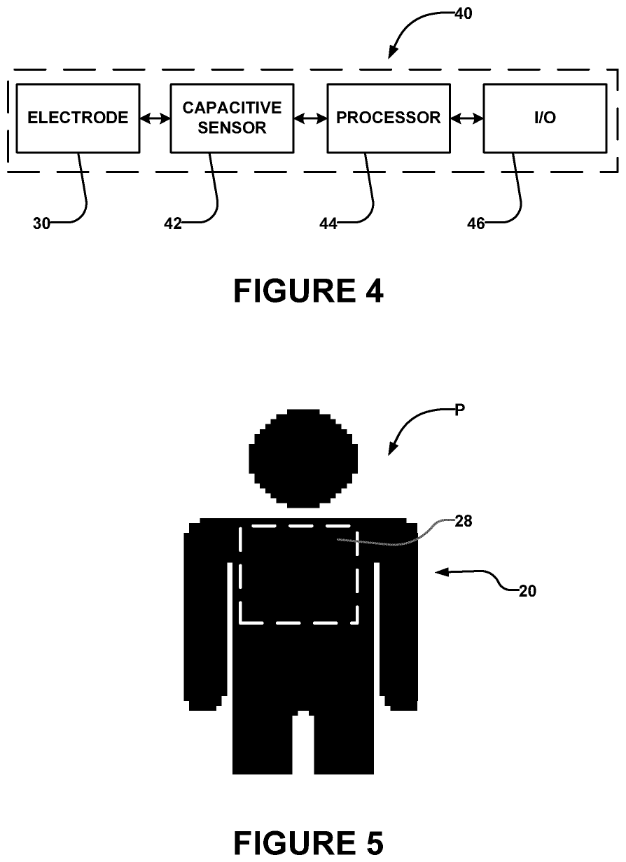

FIG. 4 shows schematically an example capacitive sensing circuit 40. In example circuit 40, electrode 30 is electrically coupled to an input of capacitive sensor 42. Electrode 30 may, for example, be coupled to the input of capacitive sensor 42 using an electrically conductive cable or wire. In some embodiments, the conductive cable or wire is a shielded cable. In some embodiments, the shielded cable is a coaxial cable.

Capacitive sensor 42 measures changes in capacitance of a capacitor formed between electrode 30 and an electrically conductive object adjacent to electrode 30 (e.g. capacitor 32 described elsewhere herein).

The electrically conductive cable or wire may be coupled to electrode 30 using any suitable method of forming an electrical connection. In preferred embodiments, the electrically conductive cable or wire is directly soldered to electrode 30. In embodiments where the electrically conductive cable or wire comprises a shielded cable, the shielded cable is preferably electrically coupled to electrode 30 in a manner that does not compromise any shielding provided by the cable (for example, the shielded cable may be soldered to electrode 30).

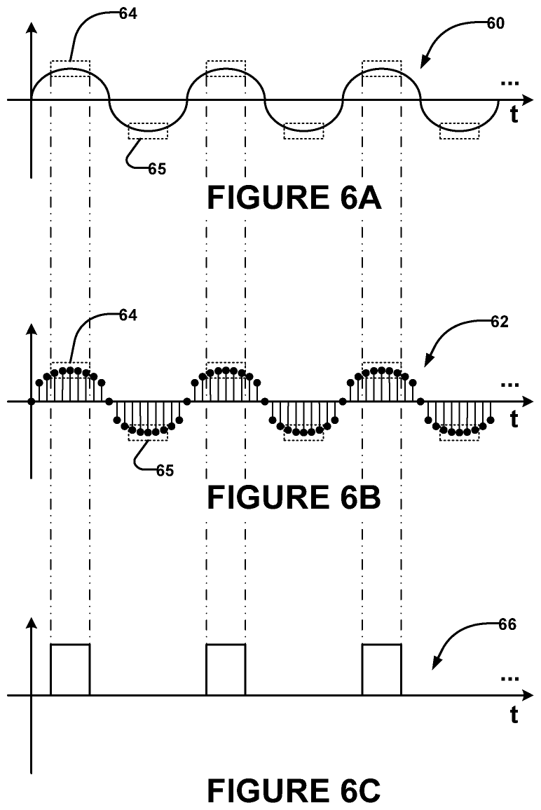

In some embodiments, capacitive sensor 42 continuously samples electrode 30. FIG. 6A illustrates an example continuous time signal 60 representative of measured capacitance C(t).

In some embodiments, capacitive sensor 42 samples electrode 30 at a sampling frequency. It is desirable that the sampling frequency be significantly higher than an expected respiration rate. The normal respiration rate for an adult human is 12-18 breaths per minute. Consequently the sampling frequency should be at least 2-3 Hz. By way of non-limiting example, capacitive sensor 42 may sample at a sampling frequency of 10 Hz, 50 Hz, 100 Hz, 200 Hz or the like. In some embodiments, capacitive sensor 42 samples at a frequency of at least 10 Hz. In some embodiments, the sampling frequency is pre-set. In some embodiments, the sampling frequency is dynamically adjusted. In some embodiments, processor 44 dynamically adjusts the sampling frequency. FIG. 6B illustrates an example discrete time signal 62 representative of measured capacitance C[n] acquired by sampling electrode 30 at the sampling frequency.

In some embodiments, capacitive sensor 42 samples electrode 30 at a first frequency while a patient P is absent (e.g. a “sleep” and/or “passive” mode). Upon a patient P being present, capacitive sensor 42 may be configured to switch to a second frequency (e.g. a “sensing” and/or “active” mode). Switching from the first sampling frequency to the second sampling frequency may, for example, be based on capacitive sensor 42 measuring a different baseline capacitance (e.g. patient P being present may vary the baseline capacitance measured by capacitive sensor 42).

Capacitive sensor 42 may be auto-calibrating. In some embodiments, capacitive sensor 42 is a commercially available capacitive sensor (one example of which is the MPR121 Proximity Capacitive Touch Sensor Controller manufactured by NXP Semiconductors Inc. of Eindhoven, Netherlands (formerly Freescale Semiconductor Inc.)).

In some embodiments, capacitive sensor 42 comprises one or more pre-processing and/or post-processing modules. Such modules may process a signal from electrode 30 and/or a signal generated by capacitive sensor 42 corresponding to a measured capacitance by filtering, averaging, down-sampling and/or the like. In some embodiments, such modules condition a signal from electrode 30 and/or a signal generated by capacitive sensor 42 to isolate and/or improve quality of components of the signal corresponding to respiration of a patient.

Sensing circuit 40 may apply any suitable method for monitoring capacitance of the system made up of an electrode and the body of a patient to detect respiratory motions. For example, changes in capacitance may be measured by detecting changes in behaviour (e.g. frequency) of a circuit such as an oscillator which includes the capacitance. As another example, capacitance may be measured by monitoring changes in electrical potential which result from charge being transferred to or from electrode 30. Capacitance may be monitored using any other presently known or future developed technology for monitoring capacitance. In some embodiments, the capacitance is measured in a way which avoids delivering high frequency signals to electrode 30. For example, sensing circuit 40 may use a direct current (DC) signal applied to electrode 30 for capacitance measurements.

In some embodiments, capacitive sensor 42 is calibrated to account for leakage of electric charge from electrode 30 (i.e. a baseline electric charge value is adjusted to account for electric charge leakage). In some embodiments, processor 44 instructs capacitive sensor 42 to calibrate. In some embodiments, capacitive sensor 42 automatically calibrates itself (i.e. capacitive sensor 42 is an auto-calibrating sensor). Capacitive sensor 42 may, for example, be calibrated periodically, upon a baseline electric charge value varying past a threshold amount, upon a practitioner initiating calibration (e.g. using GUI 200 described elsewhere herein) and/or the like.

Processor 44 receives one or more measured capacitance values from capacitive sensor 42. Processor 44 may generate a control signal 66 (see e.g. FIG. 6C) for inhibiting or enabling delivery of radiation to patient P. In some embodiments, processor 44 generates a control signal 66 that selectively enables or inhibits a radiation delivery device and/or radiation system (e.g. radiation delivery device 110 described elsewhere herein) to deliver radiation to patient P when the measured capacitance satisfies a criterion. For example, the criterion may be that the value of the measured capacitance is within a threshold window. In some embodiments, processor 44 generates a control signal 66 enabling a radiation delivery device to deliver radiation when chest 20 is expanded (i.e. threshold window 64). In some embodiments, processor 44 generates a control signal 66 enabling a radiation delivery device to deliver radiation when chest 50 is contracted (i.e. threshold window 65). FIG. 6C illustrates an example control signal 66 enabling delivery of radiation when measured capacitance is within threshold window 64.

Since the capacitance value that corresponds to any particular amplitude of respiration depends upon the position of electrode 30 relative to the patient, how deeply the patient is breathing and other factors, the particular capacitance values that correspond to a gating window will vary from patient to patient or even from one session to another with the same patient. Thresholds may be set in various ways. These include without limitation:
a processor such as processor 44 may perform peak detection to detect capacitance values corresponding to full inhalation and full exhalation. The processor may set gating thresholds automatically based on the detected capacitance values;
a technician or other user may observe a trace of the patient's respiration on a display and may set gating thresholds based on observation of the trace and/or may adjust automatically set thresholds based on observation of the trace.
   In any embodiment the thresholds may be set based on capacitance values determined while the patient is breathing according to instructions. For example, a patient may be instructed to take a deep breath and to hold. The capacitance value while holding may be determined by processor 44 and a threshold may be set based at least in part on that value.

As described elsewhere herein, delivery of radiation may also be gated based on a phase (or phases) of the patient's breathing. For example, delivery of radiation may be enabled only while patient P is inhaling. Other non-limiting example phases of breathing include a phase corresponding to patient P holding an inhaled breath (e.g. for DIBH), a phase corresponding to patient P exhaling, a phase corresponding to patient P holding an exhaled breath (e.g. for DEBH) and/or the like. Additionally, or alternatively, phases of breathing may be selected to correspond to more specific breathing events. For example, a phase may correspond to the patient's inhaling of air but only after the patient's lungs have already been partially expanded (i.e. the patient has already partially inhaled).

A patient's breathing is periodic (i.e. inhalation is followed by exhalation which is followed by inhalation and so on). In some embodiments, periodicity of the patient's breathing is determined prior to setting a gating window. For example, a system may detect features such as peaks that can be associated to specific phases of respiration and may determine a period of respiration by measuring times between detecting the features.

In some embodiments, processor 44 determines the periodicity of the patient's breathing. Processor 44 may optionally maintain an estimated phase angle of the patient's breathing based on the periodicity of the patient's breathing and the time since a reference state (e.g. fully inhaled or fully exhaled) was detected. The estimated phase may be specified in degrees or any other convenient units. In an example embodiment a gating window is defined in terms of estimated phases (e.g. at full inhalation ±a set number of degrees (where 360 degrees is a full respiration cycle).

A gating window based on a phase of the patient's breathing may be set based on measurable trends in capacitance values. If, for example, radiation is to be delivered while patient P is inhaling, a gating window may be set to enable delivery of radiation while measured capacitance values are increasing (i.e. patient P's body is moving towards electrode 30). As another example, if radiation is to be delivered while patient P is holding an inhaled breath, a gating window may be set to enable delivery of radiation while measured capacitance values remain substantially constant following a series of measured increases in capacitance value.

In some embodiments, a gating window based on a phase of the patient's breathing is at least partially set using measured capacitance values from previous respiration cycles. In such embodiments, the patient's breathing may be continuously or frequently measured. Measured capacitance values may be used to generate a signal representative of an average respiratory cycle for the patient. The average respiratory cycle may be used to predict when a desired breathing phase may occur. In some embodiments, a set number of previous respiration cycles are averaged (e.g. 2 to 10 preceding respiration cycles).

In some embodiments, a gating window is based on both an amplitude and phase of the patient's breathing. Over the course of a treatment session, patient P's breathing may vary. For example, patient P's breathing may occasionally comprise shallow breaths. A gating window may, for example, be configured to enable delivery of radiation only if both a desired phase of breathing and a threshold capacitance value is measured (e.g. the gating window may be configured to enable radiation only if the patient is within ±10 degrees (or another set range) of full inhalation and the amplitude of inhalation is at least a threshold value).

In some embodiments, a gating window specifies a single threshold gating condition to be satisfied for delivery of radiation to be enabled. In some embodiments, a gating window specifies a plurality of threshold gating conditions to be satisfied for delivery of radiation to be enabled.

I/O interface 46 is coupled to processor 44. I/O interface 46 may comprise one or more output devices which permit data (e.g. measured capacitance data) to be output to a user such as, for example, one or more displays, a printer, a speaker and/or the like. In preferred embodiments, generated gating signal 66 may be directly communicated to commercially available radiation treatment systems and/or radiation delivery devices (i.e. gating signal need not be conditioned prior to being received by a commercially available radiation treatment system/radiation delivery device).

In some embodiments, a measured capacitance is displayed as a waveform as shown in FIG. 6A. Additionally, or alternatively, I/O interface 46 may comprise one or more input devices which permit a user to provide data (e.g. a sampling frequency, a threshold window, instructions to initialize and/or terminate one or more pre-processing or post-processing modules of capacitive sensor 42, etc.) to processor 44 such as, for example, a mouse, a keyboard, a touch-screen and/or the like. In some embodiments, I/O interface 46 comprises a network interface for communicating data to and/or from processor 44 via a suitable network.

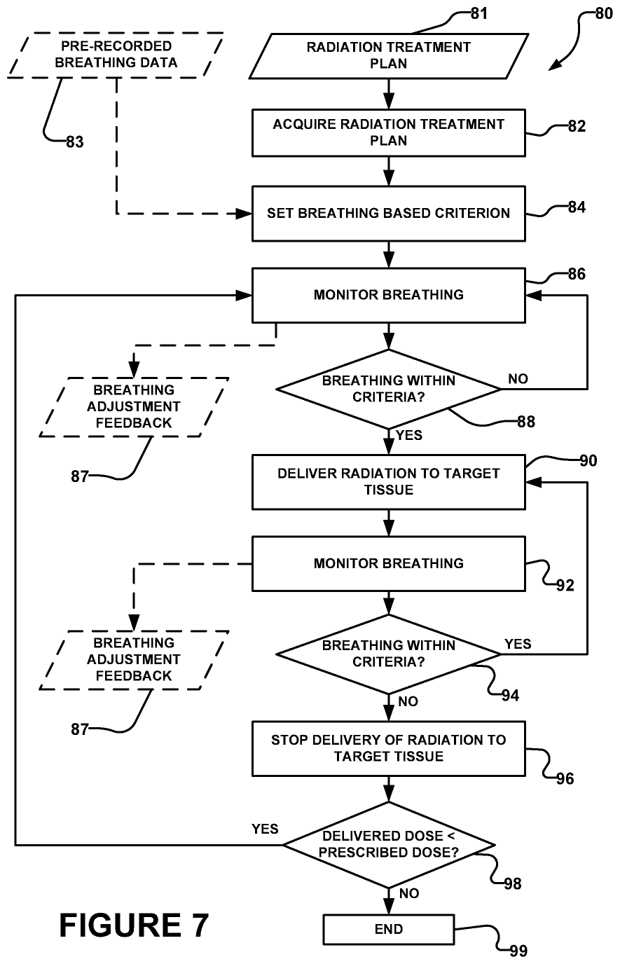

FIG. 7 is a flow chart showing at a high level an example radiation delivery control method 80.

In block 82, a radiation treatment plan 81 corresponding to patient P is acquired. Radiation treatment plan 81 specifies parameters for delivering radiation to one or more target tissues within patient P. For example, radiation treatment plan 81 may specify one or more target tissues to be radiated, locations of the one or more of the target tissues within a body of patient P, prescribed radiation dose(s), locations of sensitive non-target tissues to avoid radiating and/or the like.

In block 84, breathing based criterion for delivery of radiation is set. As described elsewhere herein, the criterion may, for example, include a threshold window of measured capacitance values for delivery of radiation (e.g. a threshold window corresponding to measured capacitance when chest 20 is expanded, a threshold window corresponding to measured capacitance when chest 20 is contracted, etc.). In some embodiments, the criterion is set at least partially using baseline breathing capacitance data measured in real time. In some embodiments, the criterion is set at least partially using pre-recorded baseline breathing capacitance data.

If patient P undergoes a pre-radiation treatment planning process, baseline breathing capacitance data 83 may be recorded during the pre-treatment process. For example, electrode 30 may be positioned adjacent to chest 20 of patient P while patient P is receiving a pre-treatment CT scan and/or being trained to breathe for the radiation treatment process. In some embodiments, pre-recorded baseline breathing capacitance data may be correlated with a generated radiation treatment plan (e.g. a radiation treatment plan may specify criterion corresponding to a breathing state for when radiation should be delivered).

In some embodiments, radiation is delivered to one or more target tissues while patient P holds their breath (i.e. a “breath hold” method of radiation therapy). In such embodiments, the block 84 criterion may correspond to a minimum amount of air that must be present in patient P's lungs for radiation to be delivered. Pre-treatment CT data in combination with capacitance values measured while patient P was trained pre-treatment to properly breath (or hold their breath) may be used to set threshold values for the block 84 criterion.

In some embodiments, the block 84 criterion gates delivery of radiation while patient p continuously breathes (i.e. a “free breathing” method of radiation therapy). In some such embodiments, the block 84 criterion may be dynamically changed to correspond to changes in patient P's breathing pattern (e.g. faster vs. slower breathes, shallow vs. fuller breaths, etc.).

In block 86, patient P's breathing is measured using contactless electrode 30. Electrode 30 may be positioned adjacent chest 20 of patient P as described elsewhere herein.

In some embodiments, a radiation treatment plan 81 is generated based on varying positions, orientations, etc. of target tissues and/or non-target tissues over the course of patient P's breathing cycle observed during pre-treatment planning sessions. If on a treatment day patient P's breathing deviates from the breathing cycle observed pre-treatment, block 86 may optionally provide feedback 87 for adjusting patient P's breathing. In some embodiments, block 86 generates audio and/or visual feedback observable directly by patient P. For example, block 86 may generate audio chirps and or visual cues using different colours, symbols, images, phrases or the like to adjust patient P's breathing. In some embodiments, block 86 provides a practitioner with feedback. The practitioner may then instruct patient P to adjust their breathing accordingly.

Block 88 determines whether the measured breathing satisfies the criterion set in block 84. If the criterion is satisfied (e.g. the measured breathing is within a threshold window), a control signal instructing a radiation delivery device and/or radiation delivery system (e.g. radiation delivery device 110 described elsewhere herein) to deliver radiation to one or more target tissues is generated in block 90. Otherwise, method 80 returns to block 86.

In some embodiments, the block 84 criterion will be satisfied if measured capacitance is above a threshold value. In some embodiments, the block 84 criterion will be satisfied if measured capacitance is below a threshold value. In some embodiments, the block 84 criterion will be satisfied if measured capacitance is between upper and lower threshold values.

In block 92, patient P's breathing is re-measured. If block 94 determines that the re-measured state of patient P's breathing continues to satisfy the block 84 criterion, method 80 returns to block 90 continuing to instruct the radiation delivery device to deliver radiation to the one or more target tissues. Otherwise, a control signal instructing the radiation delivery device to stop delivering radiation is generated in block 96.

In block 98, method 80 determines whether a prescribed dose of radiation has been delivered to the one or more target tissues. If a delivered radiation dose is less than the prescribed radiation dose, method 80 returns to block 86. In block 99, method 80 is terminated.

Another aspect of the invention provides a radiation treatment system. The radiation treatment system includes a radiation delivery device. The radiation treatment system also includes a contactless capacitive sensing system for measuring a state of a patient's breathing. Delivery of radiation by the radiation delivery device may be controlled based on the patient's measured state of breathing.

FIG. 8 shows an example radiation treatment system 100. Radiation treatment system 100 comprises radiation delivery device 110 and/or radiation delivery system 110. Radiation delivery device 110 may, for example, include gantry 112 which houses a radiation source 114 (e.g. a linear accelerator) that can be controlled to emit radiation beam 116 towards patient P. Radiation delivery device 110 may include motors to rotate gantry 112, and thereby rotate the point of origin of radiation beam 116 in an arc around patient P. Radiation delivery devices that are generally similar to device 110 are commercially available from companies such as Varian and Elekta.

Radiation treatment system 100 also includes capacitive sensing circuit 40 described elsewhere herein. In some embodiments, capacitive sensing circuit 40 is integrated into radiation delivery device 110. In some embodiments, capacitive sensing circuit 40 is independent of radiation delivery device 110. As described elsewhere herein, processor 44 may generate a control signal 66 instructing radiation delivery device 111 to start, continue or stop delivery of radiation (i.e. emission of radiation beam 116). In some embodiments, processor 44 provides control signal 66 to controller 120 of radiation delivery device 110.

In some embodiments, radiation treatment system 100 comprises data store 122. Data store 122 may record and track on a day-to-day treatment basis measured capacitance signals, measured breathing patterns, radiation treatment parameters and/or the like.

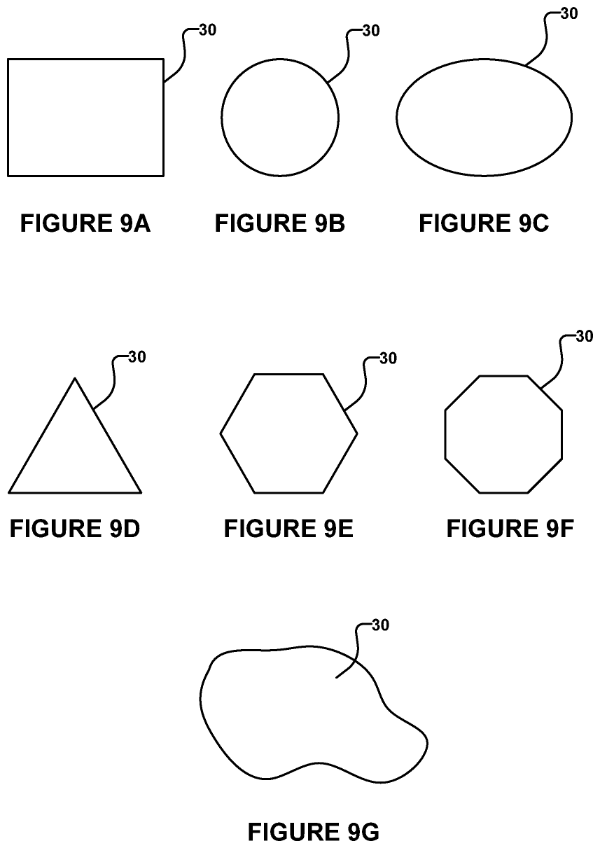

Electrode 30 may be of any shape. By way of non-limiting example, as shown in FIGS. 9A to 9F, electrode 80 may respectively be rectangular, circular, ellipsoidal, triangular, hexagonal, octagonal or the like. Electrode 30 need not be polygonal. In some embodiments, electrode 30 has a free-form shape (see e.g. FIG. 9G). In some embodiments, electrode 30 has beveled edges. In some embodiments, electrode 30 has rounded corners.

In some embodiments, electrode 30 is made of a thin film of electrically conductive material. Electrode 30 may, for example, comprise an electrically conductive material such as copper, aluminum, silver, gold or the like or an alloy of two or more of these metals.

In some embodiments, electrode 80 comprises a plurality of electrodes 31_{1,1}, . . . , 31_{i,j} (collectively electrodes 31). An electrically insulating material may electrically separate each electrode 31 from the other electrodes 31. Each electrode 31 may be separated from the other electrodes 31 by a large enough distance for a distribution of electric charge on one electrode 31 to not affect significantly a distribution of electric charge on another electrode 31. In some embodiments, each electrode 31 is separated from the other electrodes 31 by distances of at least one inch. In some embodiments, each electrode 31 is separated from the other electrodes 31 by distances of at least ¼ inch.

Different electrodes 31 positioned adjacent different regions of a patient's body may be used to measure and/or compare movement of different regions of the body. For example, a first electrode 31_{1,1} may be positioned adjacent a thoracal region of the patient and a second electrode 31_{1,2} may be positioned adjacent an abdominal region of the patient. As another example, first electrode 31_{1,1} may be positioned adjacent a left thoracal region (i.e. a region corresponding to a left lung) and second electrode 31_{1,2} may be positioned adjacent a right thoracal region (i.e. a region corresponding to a right lung).

In some embodiments, capacitive sensor 42 simultaneously measures in parallel (i.e. at the same time) first and second capacitances respectively corresponding to first and second electrodes 31_{1,1}, 31_{1,2}. In some embodiments, capacitive sensor 42 serially measures (i.e. one after the other) first and second capacitances respectively corresponding to first and second electrodes 31_{1,1}, 31_{1,2}.

Additionally, or alternatively, two or more electrodes 31 may be electrically coupled together to form a single larger electrode 30. Electrically coupling two or more electrodes 31 allows for surface area of electrode 30 to be dynamically adjusted in real time. For example, if signal strength of a measured capacitance is low, one or more additional electrodes 31 may be coupled to increase surface area of electrode 30. Dynamically coupling two or more electrodes 31 together also allows a measured region of chest 20 to be dynamically changed.

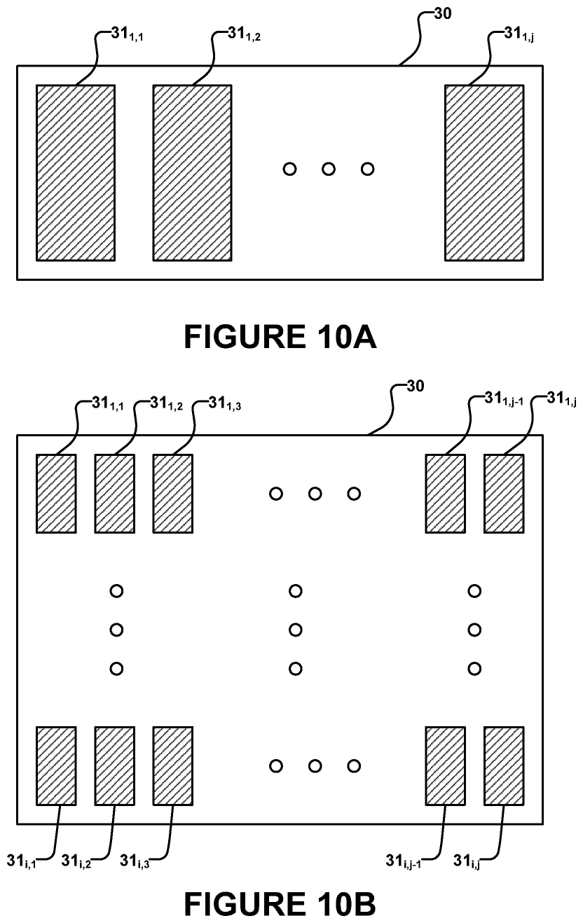

Plurality of electrodes 31 may be arranged in any pattern. Electrodes 31 may, for example, be arranged linearly (e.g. see FIG. 10A). As another example, electrodes 82 may be arranged in a checkerboard pattern (e.g. see FIG. 10B).

In some embodiments, electrode 30 comprises at least three electrodes 31. In some embodiments, electrode 30 comprises at least five electrodes 31.

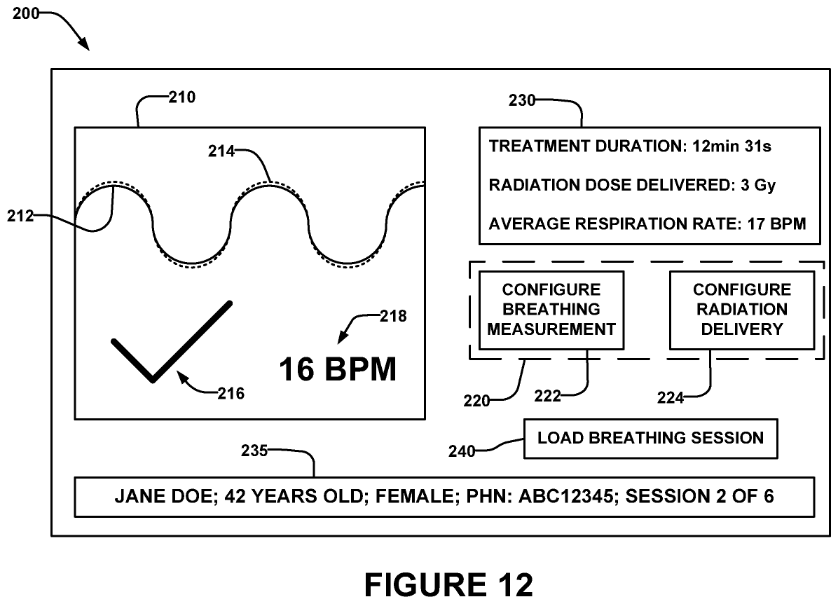

FIG. 12 schematically shows an example GUI 200. In some embodiments, GUI 200 may be used in combination with apparatus 10 and/or apparatus 100 described elsewhere herein.

GUI 200 may comprise a measured breathing module 210. Measured breathing module 210 displays parameters associated with patient P's measured breathing. In some embodiments, module 210 displays an interpolated waveform 212 of patient P's breathing. Additionally, or alternatively, module 210 may display an ideal (or averaged past) waveform 214. Additionally, or alternatively, module 210 may display alpha-numeric information 218 corresponding to patient P's measured breathing (e.g. an interpolated respiration rate per minute). Additionally, or alternatively, module 210 may display a comparison of patient 10's measured breathing with past measurements of patient 10's breathing (e.g. by overlapping interpolated waveform 212 with past breathing waveform 214). If necessary, visual cues (i.e. feedback 216) may be displayed by module 210 to aid patient P in adjusting their breathing. For example, module 210 may be green if patient P's breathing is acceptable. Conversely, module 210 may be red if patient P's breathing is not acceptable. As another example, module 210 may display a check-mark (see e.g. feedback 216) if patient P's breathing is acceptable and an X (not shown) if patient P's breathing is not acceptable.

In some embodiments, measured breathing module 210 may be displayed to patient P using a portable display. For example, module 210 may be displayed on a display embedded in a set of glasses worn by patient P, a virtual reality headset, a portable electronic device or the like. Displaying module 210 during a course of radiation treatment may allow patient P to adjust their breathing to better conform to a breathing pattern used to generate a radiation treatment plan.

Additionally, or alternatively, GUI 200 may comprise parameter setting module 220. Parameter setting module 220 allows a user to adjust one or more parameters associated with measuring patient P's breathing and/or delivering radiation. For example, module 220 may be used to vary a sampling frequency of capacitive sensor 42 described elsewhere herein (e.g. using breathing measurement parameter configuration module 222). In some embodiments, a user can use module 220 to vary a prescribed radiation dose and/or radiation treatment plan (e.g. using radiation delivery configuration module 224).

Additionally, or alternatively, GUI 200 may comprise parameter display module 230. Parameter display module 230 may display statistics associated with a course of radiation treatment. For example, module 230 may display a treatment duration, total dose delivered over the course of treatment, total dose delivered today, average respiratory rate, a metric representing uniformity of patient P's breathing or the like.

In some embodiments, GUI 200 comprises particulars module 235. For example, module 235 may display patient P's name, age, sex, personal health number and/or the like.

In some embodiments, patient P's previously recorded respiration cycles (a “breathing session”) may be retrieved using breathing session module 240. In some embodiments, patient P's retrieved breathing session may be displayed using measured breathing module 210.

In some embodiments, one or more conductive electrodes may be placed on a body of the patient. In such embodiments, capacitance may be measured between electrode 30 and the one or more conductive electrodes placed on the patient's body.

In some embodiments, apparatus and methods described herein are used to gate imaging of a patient. In such embodiments, imaging of the patient may be gated to acquire image data corresponding to desired events or phases of the patient's respiration cycle. In some embodiments, imaging is gated to acquire a series of images corresponding to the same event of the patient's respiratory cycle. The series of images may be combined to generate a single image. For example, cone beam CT imaging (which may take about a minute of imaging to acquire a useable data set) may be gated so that the patient is imaged only while the patient has fully inhaled (i.e. imaging is gated to acquire a minute's worth of data only when the patient has fully inhaled).

### Example Use Scenario

Prior to receiving radiation therapy, patient P may undergo pre-treatment analysis. The pre-treatment analysis may include developing a radiation treatment plan for the radiation therapy. This may include ascertaining locations of target and/or non-target tissues within patient P's body. Locations of tissues may be ascertained by imaging regions of interest of patient P (e.g. patient P's thorax). For example, patient P may undergo one or more CT scans, MRI scans or the like.

The pre-treatment analysis may also include training patient P to breath in a desired manner. For example, a practitioner may instruct patient P on preferred breathing techniques for DIBH. The practitioner may instruct patient P how much to breath in, how long to hold their breath for following breathing in, how quickly to exhale, etc. In some cases, a practitioner may instruct patient P on how to maintain a consistent breathing pattern (e.g. for free-breathing gating). In some cases, the practitioner may instruct patient P on how to increase or decrease their breathing rate (e.g. patient P may need to decrease their breathing rate if they start hyperventilating once treatment begins).

In one example case, patient P's breathing is measured during the pre-treatment analysis (e.g. using capacitance measuring circuit 40 described elsewhere herein). Measuring patient P's breathing may provide feedback to the practitioner and/or patient P. For example, the practitioner and/or patient P may receive sensory feedback on how patient P's breathing compares to an ideal breathing pattern. Measuring patient P's breathing may also provide patient P with dynamic guidance to assist patient P with learning a proper breathing technique. For example, patient P may receive a visual and/or audio indicator indicating when patient P has inhaled sufficiently. Patient P may then receive a countdown indicating how much longer patient P is to hold their breath for. As another example, a breathing tempo (visual and/or audio) may be provided to patient P.

Measuring patient P's breathing may also allow observation of how locations of target and/or non-target tissues change relative to patient P's breathing pattern. For example, imaging patient P while simultaneously measuring patient P's breathing may produce an image data set correlated to patient P's breathing events (e.g. a 4D CT data set). A radiation treatment plan may be tailored based on tissue locations relative to patient P's breathing. In some cases, threshold windows (e.g. for gating a radiation delivery device) may be set based on patient P's breathing that is observed during the pre-treatment analysis.

Patient P's pre-treatment analysis breathing pattern may be recorded in a data store for reference during delivery of the radiation treatment (hereinafter patient P's “baseline” breathing).

In some cases, an apparatus used to measure patient P's baseline breathing is re-located to patient P's treatment room once a course of radiation therapy is commenced. In some cases, a different apparatus is used to measure patient P's baseline breathing pattern than their treatment-day breathing pattern.

Upon patient P arriving to a clinic (or other treatment centre) for their radiation treatment, a practitioner may retrieve patient P's baseline breathing. As described elsewhere herein, patient P may be provided with feedback on how their current breathing corresponds to their baseline breathing. Additionally, or alternatively, a practitioner may (by way of non-limiting example):
instruct patient P on how they should modify their current breathing for it to correspond to their baseline breathing;
adjust a threshold window for gating delivery of radiation (or imaging) (e.g. using GUI 200 described elsewhere herein);
adjust a course of radiation therapy to be administered (e.g. using GUI 200 described elsewhere herein);
recommend re-scheduling treatment for another day.

### Interpretation of Terms

Unless the context clearly requires otherwise, throughout the description and the
“patient” is to be construed as inclusive of both human patients as well as non-human animal patients;
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Electrical circuits in various embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”)). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device for gating radiation delivery and/or monitoring respiration of a patient may implement methods as described herein by executing software instructions in a program memory accessible to the processors.

In cases where processes or blocks are presented in a given order, alternative examples may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed in parallel, or may be performed at different times.

In addition, while elements are at times shown as being performed sequentially, they may instead be performed simultaneously or in different sequences. It is therefore intended that the following claims are interpreted to include all such variations as are within their intended scope.

Some aspects of the invention may also be provided in the form of a program product. The program product may comprise any non-transitory medium which carries a set of computer-readable instructions which, when executed by a data processor, cause the data processor to execute a method of the invention. Program products according to aspects of the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

In some embodiments, the invention may be implemented in software. For greater clarity, “software” includes any instructions executed on a processor, and may include (but is not limited to) firmware, resident software, microcode, and the like. Both processing hardware and software may be centralized or distributed (or a combination thereof), in whole or in part, as known to those skilled in the art. For example, software and other modules may be accessible via local memory, via a network, via a browser or other application in a distributed computing context, or via other means suitable for the purposes described above.

Where a component (e.g. a processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

Various features are described herein as being present in “some embodiments” or “in an embodiment”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art and practicing the claimed invention, from a study of the drawings, the disclosure, and the appended claims.

In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. An apparatus for gating delivery of radiation by a radiation delivery system to a patient, the apparatus comprising:
at least one electrode positionable adjacent to but not touching a patient;
at least one capacitance sensor electrically connected to the at least one electrode and configured to monitor a capacitance of the at least one electrode and generate an output signal indicative of the capacitance; and
at least one processor configured to:
receive and process the output signal;
determine at least one of a computed measure of amplitude or phase of respiration of the patient; and
generate a gating signal for enabling or inhibiting delivery of radiation by the radiation delivery system based on at least one of the determined measure of amplitude or phase of respiration of the patient.

2. The apparatus according to claim 1, wherein the at least one processor is configured to compare the measure of amplitude of the respiration of the patient to a threshold and to generate the gating signal based at least in part on whether the amplitude is greater than the threshold.

3. The apparatus according to claim 1, wherein the at least one processor is configured to compare the measure of amplitude of the respiration of the patient to an amplitude range and to generate the gating signal based at least in part on whether the amplitude is within the amplitude range.

4. The apparatus according to claim 1, wherein the at least one processor is configured to process the output signal to determine the measure of phase of respiration of the patient, to compare the determined measure of phase to a phase window, and to generate the gating signal based at least in part on whether the measure of phase is within the phase window.

5. The apparatus according to claim 1, wherein the at least one electrode is essentially transparent to radiation deliverable by the radiation delivery system.

6. The apparatus according to claim 1, wherein the at least one electrode is supported on a substrate.

7. The apparatus according to claim 6, wherein the substrate is essentially transparent to radiation deliverable by the radiation delivery system.

8. The apparatus according to claim 6, wherein the substrate is made of a carbon fiber composite or a plastic.

9. The apparatus according to claim 1, wherein the at least one electrode is supported by a support structure that is affixable to a couch of the radiation delivery system.

10. The apparatus according to claim 9, wherein the support structure comprises index features configured to engage index elements on the couch.

11. The apparatus according to claim 9, wherein the support structure comprises a cantilever or bridge configured to extend transversely across the couch and the at least one electrode is supported by the cantilever or bridge.

12. The apparatus according to claim 1, wherein the at least one electrode comprises a plurality of electrically conductive pads and a switching network configured to electrically connect selected ones of the conductive pads in parallel to provide the at least one electrode.

13. A method for gating delivery of radiation to a patient by a radiation delivery system, the method comprising:
positioning at least one electrode adjacent to a part of a body of the patient that moves in response to respiration of the patient, the at least one electrode being spaced apart from the part of the body to form an electrical capacitor;
monitoring respiration of the patient by monitoring changes in a capacitance of the capacitor; and
based on the monitored respiration generating a gating signal, the gating signal indicating to enable or inhibit delivery of radiation by the radiation delivery system.

14. The method according to claim 13, further comprising monitoring changes in the capacitance by sensing the capacitance at a sampling frequency.

15. The method according to claim 14, wherein the sampling frequency is in a range between 2 Hz to 200 Hz.

16. The method according to claim 13, wherein monitoring the changes in the capacitance comprises processing an output signal from a capacitive sensor and conditioning the output signal to isolate signal components corresponding to respiration of the patient.

17. The method according to claim 13, further comprising generating feedback for the patient to assist the patient in controlling the patient's breathing by providing the patient with one or more indicators based on an output signal of a capacitive sensor.

18. The method according to claim 17, wherein the feedback comprises at least one of:
showing the patient by how much their respiration varies from a desired respiration,
reminding the patient to hold their breath at a desired point,
showing the patient if they are successfully holding their breath, and
letting the patient know for how much longer they are expected to hold their breath.

19. The method according to claim 17, further comprising displaying the feedback using at least one of a countdown timer display, a bar indicator, a color changing indicator, one or more displays embedded in a set of glasses worn by the patient, a virtual reality head set, a portable electronic device, and a display mounted within a field of view of the patient.

20. The method according to claim 13, further comprising generating the gating signal based on one or both of an amplitude and a phase of the monitored respiration.

21. The method according to claim 20, further comprising determining the phase of the monitored respiration by tracking one or more trends in an output signal of the capacitive sensor.

22. The method according to claim 13, further comprising setting a gating window based on previously acquired respiration data of the patient.

23. The method according to claim 13, wherein generating the gating signal comprises determining whether at least one of a phase or an amplitude of the monitored respiration is within a gating window and dynamically adjusting the gating window based on the monitored respiration.

24. The method according to claim 13, wherein the at least one electrode is positioned at a minimum distance of 1 cm to 10 cm away from the part of the body of the patient.

25. The method according to claim 13, further comprising varying a signal strength of the monitored respiration by adjusting one or both of a distance of the at least one electrode from the body of the patient and an angle of the at least one electrode relative to the body of the patient.

26. The method according to claim 13, further comprising indexing a support structure that supports the at least one electrode to a couch of the radiation delivery system.

27. The method according to claim 26, wherein the support structure comprises a substrate that extends transversely to the body of the patient, wherein the at least one electrode is supported by the substrate.

28. The method according to claim 13, further comprising placing the at least one electrode in a location within an area corresponding to a path of a radiation beam wherein the at least one electrode and the substrate are essentially transparent to radiation of the radiation beam.

29. The method according to claim 13, wherein monitoring changes in the capacitance of the capacitor comprises applying a direct current (DC) signal to the at least one electrode.

30. The method according to claim 13, wherein the at least one electrode comprises a plurality of electrodes, each of the plurality of electrodes located proximate to a different part of the body.

31. The method according to claim 30, further comprising electrically connecting a plurality of the electrodes in parallel.

32. The method according to claim 13, further comprising placing a conductive material on a surface of the body opposite the at least one electrode.
